(19) United States
(12) Patent Application Publication
(10) Pub. No.: US 2003/0045680 A1
(43) Pub. Date: Mar. 6, 2003

(54) Peptidic modulators of the androgen receptor

(75) Inventors: John L. Joyal, Melrose, MA (US); John Mueller, Medford, MA (US); Vibha B. Oza, Lexington, MA (US); Mark A. Findeis, Belmont, MA (US)

Correspondence Address: LAHIVE & COCKFIELD, 28 STATE STREET, BOSTON, MA, 02109, US

(73) Assignee: Praecis Pharmaceuticals Inc., Waltham, MA (US)

(21) Appl. No.: 10/097,175

(22) Filed: Mar. 12, 2002

Related U.S. Application Data
(60) Provisional application No. 60/275,240, filed on Mar. 12, 2001.
(60) Provisional application No. 60/352,399, filed on Jan. 28, 2002.

(51) Int. Cl. C07K 7/08; C07K 7/06

(52) U.S. Cl. 530/327; 530/328

(57) ABSTRACT

The present invention provides peptide compounds which bind to the androgen receptor. In preferred embodiments, the peptide compounds of the invention inhibit binding of the androgen receptor DNA binding domain to DNA, in particular, the androgen responsive elements. These compounds are useful for the treatment of androgen-associated disorders including, for example, prostate cancer.

### RELATED APPLICATION

This application claims priority to U.S. Provisional Patent Application Serial No. 60/275,240 filed Mar. 12, 2001, and U.S. Provisional Patent Application Serial No. 60/352,399 filed Jan. 28, 2002, the entire contents of each of which are incorporated herein by reference.

### BACKGROUND OF THE INVENTION

The androgen receptor is a ligand-activated nuclear transcription factor and is a member of the nuclear receptor superfamily. The receptor is a protein of 919 amino acid residues with a molecular weight of 98.4 kDa and comprises four domains: (1) an N-terminal domain involved in transcriptional regulation, (2) a DNA-binding domain, (3) a hinge region and (4) a C-terminal ligand binding domain. (O'Malley B. (1990) Mol. Endocrinology 4:363-369). The two primary ligands for the androgen receptor are testosterone and dihydrotestosterone. Binding of a ligand to the androgen receptor induces a number of structural changes within the receptor and ultimately leads to dimerization and binding to promoter DNA sequences associated with particular genes. The range of genes activated by the androgen receptor is tissue-specific, with such inter-tissue differences attributable to differences in androgen receptor expression and tissue-specific expression of co-activators.

The androgen receptor has been shown to be present in non-malignant prostate tissues, benign prostatic hyperplasia tissues, and primary and metastatic prostate cancers. Testosterone is a known growth factor for prostate cancer, and surgical or medical androgen ablation is the current standard for treating advanced prostate cancer. The androgen receptor is an indirect target of this therapy, which seeks to down-regulate androgen receptor mediated gene transcription by depriving the receptor of its activation ligands.

Although reduction of testosterone to castrate levels results in prostate tumor regression, many patients ultimately become unresponsive to hormonal therapy, presumably due to mutations of the androgen receptor which alter its ligand binding specificity. For example, the LNCaP cell line, which is derived from a lymph node deposit of a hormonally nonresponsive metastatic prostate cancer, is responsive to androgens, estrogen, progesterone and flutamide, an anti-androgen, despite having no estrogen or progesterone receptors. (Berrevoets C. A. et al. (1993) J. Steroid Biochem. Mol. Biol. 46:731-736). This suggests that the androgen receptor in these cells has expanded its repertoire of ligands beyond the native repertoire of ligands. Analysis of the androgen receptor gene from this cell line revealed a single point mutation. (Veldscholte J. et al. (1990) Biochem. Biophys. Res. Comm. 173:534-540). A mutant receptor has also been observed in up to half of hormone-relapsed prostate cancer cells. (Taplen, M. E. et al. (1995) New England J. Med. 332:1393-98). Increased production of androgen receptor has also been observed in a significant number of recurrent prostate tumors. These findings suggest that, in certain cases, upregulation of receptor production may increase sensitivity to the residual levels of androgen remaining during hormone therapy.

One approach to treating hormonally non-responsive prostate cancer is direct inhibition of the androgen receptor. Accordingly, there are needed therapeutic agents and methods of use thereof which directly inhibit the androgen receptor, e.g., which inhibit androgen receptor mediated gene transcription.

### SUMMARY OF THE INVENTION

The present invention provides peptide compounds which bind to the androgen receptor. In preferred embodiments, the peptide compounds of the invention inhibit binding of the androgen receptor DNA binding domain to DNA, in particular, the androgen responsive elements. The peptide compounds of the invention are useful in, for example, the treatment of prostate cancer, e.g., hormonally refractive prostate cancer.

In one aspect, the invention provides peptide compounds of the formula

Y₁-(Xₐ)ₖ-(X₁¹)ₘ-(X₂¹)ₙ-(X₃¹)ₚ-(X₄¹)-(X₅¹)-(X₆¹)-(X₇¹)_{q}-(X₈¹)ᵣ-(X_{b})ₛ-Y₂ (I),

wherein Y₁ is hydrogen, alkyl or acyl and Y₂ is —OH or —NR₂, where each R is independently hydrogen or lower alkyl. Xₐ and X_{b} are each, independently, a peptidic structure comprising from 1 to 25 amino acid residues. X₁¹ is lysine, alanine, threonine, histidine, methionine or an analogue thereof; X₂¹ is threonine, glutamic acid, alanine, isoleucine, valine or an analogue thereof; X₃¹ is glutamic acid, alanine, proline, threonine or aspartic acid or an analogue thereof; X₄¹ is serine, valine, glutamine or alanine or an analogue thereof; X₅¹ is serine, alanine, phenylalanine, leucine, valine or an analogue thereof; X₆¹ is serine or an analogue thereof; X₇¹ is aspartic acid, glutamic acid, alanine, methionine, proline, valine or an analogue thereof; and X₈¹ is serine, threonine, phenylalanine, glutamic acid, isoleucine or an analogue thereof. k, m, n, p, q, r and s are each, independently, 0 or 1. Preferably, when k is 1, m, n and p are each 1. Preferably, when m is 1, n and p are each 1. Preferably, when n is 1, p is 1. Preferably, when s is 1, q and r are each 1. Preferably, when r is 1, q is 1.

Peptide compounds of formula I also include peptides where one of the amino acid residues identified for X₁¹-X₈¹ is conservatively substituted.

In another aspect, the invention provides peptide compounds of the formula

Y₁-Xₐ-X₁²-X₂²-X₃²-X₄²-X₅²-X₆²-X₇²-X_{b}-Y₂ (II)

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl; Xₐ and X_{b} are each, independently, a direct bond or a peptidic structure comprising from 1 to about 25 amino acid residues; X₁² is lysine, serine, phenylalanine, glycine or serine; preferably glycine, phenylalanine or serine, most preferably serine; X₂² is serine, tyrosine, valine, alanine or glycine, preferably serine, tyrosine or valine; X₃² is any amino acid residue, but is preferably proline, isoleucine, serine, tryptophan, alanine, valine, tyrosine, or phenylalanine; X₄² is serine, leucine, tyrosine, phenylalanine, asparagine or alanine, preferably serine or leucine; X₅² is leucine, serine, phenylalanine or tyrosine, preferably leucine or serine, most preferably leucine; X₆² is tryptophan; and X₇² is proline.

In one embodiment, the peptide compound comprises the structure: Y₁-Xₐ-Ser-Ser-X₃²-Ser-Leu-Trp-Pro-X_{b}-Y₂; Y₁-Xₐ-Ser-Tyr-X₃²-Ser-Leu-Trp-Pro-X_{b}-Y₂; Y₁-Xₐ-Ser-Val-X₃¹-Ser-Leu-Trp-Pro-X_{b}-Y₂; Y₁-Xₐ-Ser-Ser-X₃²-Leu-Leu-Trp-Pro-X_{b}-Y₂; Y₁-Xₐ-Ser-Tyr-X₃²-Leu-Leu-Trp-Pro-X_{b}-Y₂; or Y₁-Xₐ-Ser-Val-X₃²-Leu-Leu-Trp-Pro-X_{b}-Y₂ In another embodiment, the peptide compound comprises the structure: Y₁-Xₐ-Gly-Ser-Pro-Ala-Leu-Trp-Pro-X_{b}-Y₂; Y₁-Xₐ-Leu-Val-Ile-Asp-Leu-Trp-Pro-X_{b}-Y₂; Y₁-Xₐ-Ser-Tyr-Ser-Phe-Leu-Trp-Pro-X_{b}-Y₂; Y₁-Xₐ-Ser-Tyr-Trp-Tyr-Leu-Trp-Pro-X_{b}-Y₂; Y₁-Xₐ-Phe-Tyr-Ala-Ser-Leu-Trp-Pro-X_{b}-Y₂; Y₁-Xₐ-Tyr-Ala-Ala-Ser-Phe-Trp-Pro-X_{b}-Y₂; Y₁-Xₐ-Phe-Ala-Val-Leu-Ser-Trp-Pro-X_{b}-Y₂; Y₁-Xₐ-Gly-Val-Tyr-Leu-Ser-Trp-Pro-X_{b}-Y₂; Y₁-Xₐ-Ser-Ser-Trp-Leu-Ser-Trp-Pro-X_{b}-Y₂; or Y₁-Xₐ-Ser-Gly-Phe-Ser-Tyr-Trp-Pro-X_{b}-Y₂.

Preferably, the peptide compound comprises the structure: Ser-Met-Phe-Gly-Ser-Pro-Ala-Leu-Trp-Pro-Leu-Arg (SEQ ID NO:56); Leu-Val-Ile-Asp-Leu-Trp-Pro-Ala-Phe-Val-Arg (SEQ ID NO:57); Ser-Tyr-Ser-Phe-Leu-Trp-Pro-Ile-Val-Phe-Lys (SEQ ID NO:58); Glu-Leu-Phe-Ser-Tyr-Trp-Pro-Trp-Pro-Phe-Tyr-Arg (SEQ ID NO:59); Phe-Tyr-Ala-Ser-Leu-Trp-Pro-His-Leu-Tyr-Val (SEQ ID NO:60); Val-Gly-Thr-Gly-Ala-Ser-Ser-Tyr-Ala-Ala-Ser-Phe-Trp-Pro-Trp-Met-Thr-Tyr-Tyr-Trp (SEQ ID NO:61); Phe-Ala-Val-Leu-Ser-Trp-Pro-Leu-Tyr-Glu-Tyr (SEQ ID NO:62); Gly-Val-Tyr-Leu-Ser-Trp-Pro-Gly-Ser-Met-Ala (SEQ ID NO:63); Ser-Ser-Tyr-Ser-Ser-Trp-Leu-Ser-Trp-Pro-Arg (SEQ ID NO:64); or Ser-Gly-Phe-Ser-Tyr-Trp-Pro-Phe-Phe-Phe-Val (SEQ ID NO:65).

In yet another aspect, the invention provides peptide compounds of the formula

Y₁-Xₐ-Ser-Pro-Asp-X₁³-X₂³-Ala-X_{b}-Y₂ (III),

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl; Xₐ and X_{b} are each, independently, a direct bond or a peptidic structure comprising from 1 to about 25 amino acid residues; X₁³ is Tyr or Leu; and X₂³ is Gln or Leu. In one embodiment, the peptide compound comprises the structure Y₁-Xₐ-Ser-Pro-Asp-Tyr-Gln-Ala-X_{b}-Y₂ or Y₁-Xₐ-Ser-Pro-Asp-Leu-Leu-Ala-X_{b}-Y₂. Preferably the peptide compound comprises the structure Arg-Met-Val-Ser-Pro-Asp-Tyr-Gln-Ala-Thr-Ser-Pro (SEQ ID NO:66) or Leu-Ser-Phe-Ser-Pro-Asp-Leu-Leu-Ala-Leu-Arg-Gly-Met (SEQ ID NO:67).

In a further aspect, the invention provides peptide compounds of the formula

Y₁-Xₐ-Ser-Pro-Ala-Leu-Trp-X_{b}-Y₂ (IV),

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl. Xₐ and X_{b} are each, independently, a direct bond or a peptidic structure comprising from 1 to about 25 amino acid residues.

Preferably the peptide compound comprises the structure: Ser-Met-Phe-Gly-Ser-Pro-Ala-Leu-Trp-Pro-Leu-Arg (SEQ ID NO:68) or Val-Ser-Pro-Ala-Leu-Trp-Ser-Ser-Leu-Arg-Gly (SEQ ID NO:69).

In another aspect, the invention provides peptide compounds of the formula

Y₁-Xₐ-X₁⁵-X₂⁵-Trp-Leu-X₃⁵-Ser-X_{b}-Y₂ (V),

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl. Xₐ and X_{b} are each, independently, a direct bond or a peptidic structure comprising from 1 to about 25 amino acid residues; X₁⁵ is Ser or Pro, preferably Ser; X₂⁵ is Ser or Trp, preferably Ser; and X₃⁵ is Phe or Ala, preferably Ala.

In one embodiment, the peptide compound comprises the structure: Y₁-Xₐ-Ser-Ser-Trp-Leu-Ala-Ser-X_{b}-Y₂; Y₁-Xₐ-Ser-Trp-Trp-Leu-Ala-Ser-X_{b}-Y₂; or Y₁-Xₐ-Pro-Ser-Trp-Leu-Ala-Ser-X_{b}-Y₂.

Preferably the peptide compound comprises the structure: Gly Phe Val Ser Ser Trp Leu Phe Ser Ala Ser (SEQ ID NO:70); Ala Ser Met Ser Trp Trp Leu Ala Ser Ser Pro (SEQ ID NO:71); or Phe Val His Pro Ser Trp Leu Ala Ser Phe Leu (SEQ ID NO:72).

In yet another aspect, the invention provides peptide compounds of the formula

Y₁-Xₐ-Phe-X₁⁶-X₂⁶-X₃⁶-X₄⁶-X₅⁶-X₆⁶-X_{b}-Y₂ (VI),

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl; Xₐ and X_{b} are each, independently, a direct bond or a peptidic structure comprising from 1 to about 25 amino acid residues; X₁⁶ is Ser or Val, preferably Ser;

X₂⁶ is His, Thr, Gly or Phe, preferably His or Thr; X₃⁶ is Pro, Ile, or Tyr, preferably Pro; X₄⁶ is Ser, Tyr, Ala, Gly or Cys, preferably Ala; X₅⁶ is Trp or Gly, preferably Trp; and X₆⁶ is Leu, Ser, Tyr or Arg, preferably Ser.

In one embodiment the peptide compound comprises the structure: Y₁-Xₐ-Phe-Ser-His-Pro-Ala-Trp-Ser-X_{b}-Y₂; Y₁-Xₐ-Phe-Ser-Thr-Pro-Ala-Trp-Ser-X_{b}-Y₂; Y₁-Xₐ-Phe-Val-His-Pro-Ser-Trp-Leu-X_{b}-Y₂; Y₁-Xₐ-Phe-Ser-His-Pro-Tyr-Trp-Ser-X_{b}-Y₂; Y₁-Xₐ-Phe-Ser-Gly-Pro-Ala-Trp-Ser-X_{b}-Y₂; Y₁-Xₐ-Phe-Ser-Phe-Ile-Ala-Trp-Ser-X_{b}-Y₂; Y₁-Xₐ-Phe-Ser-Thr-Tyr-Gly-Trp-Tyr-X_{b}-Y₂; or Y₁-Xₐ-Phe-Ser-Thr-Pro-Cys-Gly-Arg-X_{b}-Y_{2.}

Preferably the peptide compound comprises the structure: Phe Val His Pro Ser Trp Leu Ala Ser Phe Leu (SEQ ID NO:73); Phe Ser His Pro Tyr Trp Ser Tyr Leu Phe Ser (SEQ ID NO:74); Phe Ser Gly Pro Ala Trp Ser Leu His Lys His (SEQ ID NO:75); Phe Ser Phe Ile Ala Trp Ser Pro Ala Met Leu (SEQ ID NO:76); Phe Ser Thr Tyr Gly Trp Tyr Ser Pro Phe His (SEQ ID NO:77); or Ile Pro Phe Ser Thr Pro Cys Gly Arg Trp Cys (SEQ ID NO:78).

In a further aspect, the invention provides peptide compounds of the formula

Y₁-Xₐ-X₁⁷-X₂⁷-X₃⁷-X₄⁷-X₅⁷-X₆⁷-X₇⁷-X₈⁷-X₉⁷-X_{b}-Y₂ (VII)

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl; Xₐ and X_{b} are each, independently, a direct bond or a peptidic structure comprising from 1 to about 25 amino acid residues; X₁⁷ is Ser, Trp, Phe, Ala or Val, preferably Ser; X₂⁷ is Ser, Leu, Pro, Trp or Val, preferably Ser; X₃⁷ is Ser, Tyr, Val, Met or His, preferably Val, Tyr or Ser, more preferably Tyr or Ser; X₄⁷ is Pro, Ser, Leu, Arg, Tyr, Val or Trp, preferably Pro or Ser, more preferably Pro; X₅⁷ is Phe, Ser, Trp, Arg or Leu, preferably Phe, Trp or Ser; X₆⁷ is Trp, Asn, Met, Asp, Gly, Phe, preferably Trp; X₇⁷ is Ala, Trp, Leu or Ser, preferably Leu; X₈⁷ is Arg, Pro, Phe, Ala, Ser, Tyr, preferably Ala; and X₉⁷ is Pro, Asn, Ser, Trp, Thr, Phe, Ala, preferably Ser or Pro, more preferably Ser.

In one embodiment, the peptide compound comprises the structure: Y₁-Xₐ-Ser-Ser-Ser-Pro-Trp-Trp-Leu-Ala-Ser-Xb-Y2; Y1-Xa-Ser-Ser-Ser-Pro-Phe-Trp-Leu-Ala-Ser-Xb-Y2; Y1-Xa-Ser-Ser-Ser-Pro-Ser-Trp-Leu-Ala-Ser-Xb-Y2; Y1-Xa-Ser-Ser-Tyr-Pro-Trp-Trp-Leu-Ala-Ser-Xb-Y2; Y1-Xa-Ser-Ser-Tyr-Pro-Phe-Trp-Leu-Ala-Ser-Xb-Y2; Y1-Xa-Ser-Ser-Tyr-Pro-Ser-Trp-Leu-Ala-Ser-Xb-Y2; Y1-Xa-Ser-Ser-Val-Pro-Phe-Trp-Ala-Arg-Pro-Xb-Y2; Y₁-Xₐ-Ser-Leu-Val-Pro-Phe-Asn-Trp-Pro-Asn-X_{b}-Y₂; Y₁-Xₐ-Ser-Pro-Tyr-Pro-Ser-Met-Leu-Phe-Ser-X_{b}-Y₂; Y₁-Xₐ-Trp-Trp-Tyr-Pro-Trp-Asp-Leu-Ala-Ser-X_{b}-Y₂; Y₁-Xₐ-Phe-Val-His-Pro-Ser-Trp-Leu-Ala-Ser-X_{b}-Y₂; Y₁-Xₐ-Ala-Ser-Met-Ser-Trp-Trp-Leu-Ala-Ser-X_{b}-Y₂; Y₁-Xₐ-Ser-Ser-Tyr-Ser-Ser-Trp-Leu-Ser-Trp-X_{b}-Y₂; Y₁-Xₐ-Ser-Ser-Ser-Leu-Phe-Trp-Ser-Ala-Thr-X_{b}-Y₂; Y₁-Xₐ-Ser-Ser-Ser-Arg-Ser-Trp-Ala-Ala-Phe-X_{b}-Y₂; Y₁-Xₐ-Ser-Ser-Ser-Tyr-Trp-Gly-Leu-Tyr-Pro-X_{b}-Y₂; Y₁-Xₐ-Ser-Ser-Tyr-Val-Arg-Trp-Leu-Ala-Ala-Xb-Y₂; or Y₁-Xₐ-Val-Ser-Ser-Trp-Leu-Phe-Ser-Ala-Ser-X_{b}-Y₂.

Preferably the peptide compound comprises the structure: Ser-Ser-Val-Pro-Phe-Trp-Ala-Arg-Pro-Leu-Val (SEQ ID NO:79); Ser-Leu-Ser-Ser-Leu-Val-Pro-Phe-Asn-Trp-Pro-Asn-Leu-Phe-Ser-Trp-Arg-Tyr-Ser-Trp (SEQ ID NO:80); Leu-Gly-Ser-Pro-Tyr-Pro-Ser-Met-Leu-Phe-Ser-Asp-His (SEQ ID NO:81); Phe-Trp-Trp-Tyr-Pro-Trp-Asp-Leu-Ala-Ser-Tyr (SEQ ID NO:82); Phe-Val-His-Pro-Ser-Trp-Leu-Ala-Ser-Phe-Leu (SEQ ID NO:83); Ala-Ser-Met-Ser-Trp-Trp-Leu-Ala-Ser-Ser-Pro (SEQ ID NO:84); Ser-Ser-Tyr-Ser-Ser-Trp-Leu-Ser-Trp-Pro-Arg (SEQ ID NO:85); Ser-Ser-Ser-Leu-Phe-Trp-Ser-Ala-Thr-Ser-Arg (SEQ ID NO:86); Ser-Ser-Ser-Arg-Ser-Trp-Ala-Ala-Phe-Glu-His (SEQ ID NO:87); Ser-Ser-Ser-Tyr-Trp-Gly-Leu-Tyr-Pro-Ser-Leu-Ser-Leu (SEQ ID NO:88); Ser-Ser-Tyr-Val-Arg-Trp-Leu-Ala-Ala-Ala-Gln (SEQ ID NO:89); Gly-Phe-Val-Ser-Ser-Trp-Leu-Phe-Ser-Ala-Ser (SEQ ID NO:90); or Ser-His-Gly-Trp-Phe-Trp-Ser-Ser-Ser-Gln-Gly (SEQ ID NO:91).

In another aspect, the invention provides peptide compounds of the formula

Y₁-Xₐ-Cys-X₁⁸-X₂⁸-X₃⁸-X₄⁸-Gly-X₅⁸-X₆⁸-X₇⁸-Cys-X_{b}-Y₂ (VIII),

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl; Xₐ and X_{b} are each, independently, a direct bond or a peptidic structure comprising from 1 to about 25 amino acid residues; X₁⁸ is an aromatic amino acid residue or threonine; X₂⁸ is Gly, Phe, Gln, Arg, Met, Trp; X₃⁸ is Asp or Glu; X₄⁸ is Glu or Asp; X₅⁸ is Tyr or Trp; X₆⁸ is Pro, Trp, Thr, Leu, Phe, Tyr, Met; and X₇⁸ is His, Asp, Ser, Ala, Leu, Met, Trp.

Preferably the peptide compound comprises the structure: Y₁-Xₐ-Cys-Trp-Gly-Asp-Asp-Gly-Trp-Pro-Ala-His-Cys-X_{b}-Y₂; Y₁-Xₐ-Cys-Phe-Phe-Asp-Asp-Gly-Tyr-Trp-Trp-Asp-Cys-X_{b}-Y₂; Y₁-Xₐ-Cys-Tyr-Gln-Asp-Asp-Gly-Tyr-Thr-Val-Ser-Cys-X_{b}-Y₂; Y₁-Xₐ-Cys-Tyr-Met-Glu-Asp-Gly-Tyr-Leu-Trp-Ala-Cys-X_{b}-Y₂; Y₁ -Xₐ-Cys-Phe-Phe-Glu-Asp-Gly-Tyr-Phe-His-Ala-Cys-X_{b}-Y₂; Y₁-Xₐ-Cys-Tyr-Gly-Asp-Asp-Gly-Trp-Phe-Met-Leu-Cys-X_{b}-Y₂; Y₁-Xₐ-Cys-Tyr-Met-Asp-Glu-Gly-Trp-Tyr-Tyr-Ser-Cys-X_{b}-Y₂; Y₁-Xₐ-Cys-Trp-Gln-Glu-Asp-Gly-Trp-Leu-Tyr-Leu-Cys-X_{b}-Y₂; Y₁-Xₐ-Cys-Trp-Arg-Glu-Asp-Gly-Tyr-Trp-Trp-Tip-Cys-X_{b}-Y₂; Y₁-Xₐ-Cys-Trp-Gln-Asp-Asp-Gly-Trp-Tyr-Tyr-Met-Cys-X_{b}-Y₂; Y₁-Xₐ-Cys-Tyr-Trp-Asp-Asp-Gly-Trp-Met-Leu-Glu-Cys-X_{b}-Y₂; Y₁-Xₐ-Cys-Trp-Arg-Glu-Asp-Gly-Tyr-Trp-Trp-Trp-Cys-X_{b}-Y₂; or Y₁-Xₐ-Cys-Thr-Trp-Asp-Asp-Gly-Trp-Met-Phe-Leu-Cys-X_{b}-Y₂.

Preferably, at least one of Xₐ and X_{b} comprises an amino acid sequence which facilitates transport of the peptide into mammalian cells and, preferably, into the nuclei of mammalian cells.

In another embodiment, the invention provides an assay for identifying a peptide compound which binds to the androgen receptor. This method includes contacting the androgen receptor, or a functional domain thereof, with a peptide compound of formulas I-VIII, thereby forming a peptide compound-androgen receptor complex; contacting the peptide compound-androgen receptor complex with a test compound; and determining if the peptide compound dissociates from the peptide compound-androgen receptor complex in the presence of the test compound. If the peptide is displaced from the peptide compound-androgen receptor complex, the test compound binds to the androgen receptor or androgen receptor functional domain.

In another embodiment, the present invention provides a method of treating an androgen-associated disorder in a subject. The method includes administering to the subject a therapeutically effective amount of a peptide compound of formulas I-VIII, thereby treating an androgen-associated disorder in the subject.

In yet another embodiment, the invention provides a pharmaceutical composition comprising a peptide compound of formulas I-VIII and a pharmaceutically acceptable carrier.

The present invention provides several advantages. For example, the compounds of the invention bind the DNA-binding region of the androgen receptor and, therefore, inhibit gene activation by a native androgen receptor or an androgen receptor which has been mutated in, for example, the ligand binding domain.

Other features and advantages of the invention will be apparent from the following detailed description and claims.

### DETAILED DESCRIPTION OF THE INVENTION

The present invention provides peptide compounds which bind to the androgen receptor, as well as methods of using these compounds for treating androgen-associated disorders such as prostate cancer, e.g., hormonally refractive prostate cancer. The peptide compounds of the present invention may also be used as elution reagents in assays for the identification of compounds which bind to the androgen receptor. Preferably, the peptide compounds of the invention bind to the DNA-binding domain of the androgen receptor. Without intending to be bound by theory, it is believed that the peptide compounds of the invention inhibit androgen-receptor mediated gene activation by inhibiting the binding of the androgen receptor to the androgen responsive elements of DNA (see infra).

Various aspects of the invention are described further in the following subsections.

I. Peptide Compounds of the Invention

In one aspect, the present invention provides peptide compounds of the formula

Y₁-(Xₐ)ₖ-(X₁¹)ₘ-(X₂¹)ₙ-(X₃¹)ₚ-(X₄¹)-(X₅¹)-(X₆¹)-(X₇¹)_{q}-(X₈¹)ᵣ-(X_{b})ₛ-Y₂ (I),

where Y₁ is hydrogen, alkyl or acyl and Y₂ is —OH or —NR₂, where each R is independently hydrogen or lower alkyl. Xa and X_{b} are each, independently, a peptidic structure comprising from 1 to 25 amino acid residues. X₁¹ is lysine, alanine, threonine, histidine, methionine or an analogue thereof; X₂¹ is threonine, glutamic acid, alanine, isoleucine, valine or an analogue thereof; X₃¹ is glutamic acid, alanine, proline, threonine or aspartic acid or an analogue thereof; X₄¹ is serine, valine, glutamine or alanine or an analogue thereof; X₅¹ is serine, alanine, phenylalanine, leucine, valine or an analogue thereof; X₆¹ is serine or an analogue thereof; X₇¹ is aspartic acid, glutamic acid, alanine, methionine, proline, valine or an analogue thereof; and X₈¹ is serine, threonine, phenylalanine, glutamic acid, isoleucine or an analogue thereof. K, m, n, p, q, r and s are each, independently, 0 or 1.

The peptide compounds of formula I also include peptides in which one or more of X₁¹, X₂¹, X₃¹, X₄¹, X₅¹, X₆¹, X₇¹, or X₈¹ is conservatively substituted. Any or all of X₁¹-X₈¹ can be conservatively substituted. Preferably six or fewer, five or fewer, four or fewer, three or fewer, two, one or none of X₁¹-X₈¹ are conservatively substituted. Preferably, X₆¹ is not substituted and at least one of X₄¹ and X₅¹ is serine.

In preferred embodiments of the peptide compounds of formula I, at least one of X₄¹ and X₅¹ is serine. In particularly preferred embodiments, X₁¹ is threonine or histidine; X₂¹ is threonine or isoleucine; X₃¹ is glutamic acid or aspartic acid; X₄¹ is serine; X₅¹ is serine; X₇¹ is aspartic acid and X₈¹ is glutamic acid.

In one subset of preferred peptide compounds of formula I, when k is 1, m, n and p are each 1; when m is 1, n and p are each 1; when n is 1, p is 1; when s is 1, q and r are each 1; and when r is 1, q is 1.

In another aspect, the invention provides peptide compounds of the formula

Y₁-Xₐ-X₁²-X₂²-X₃²-X₄²-X₅²-X₆²-X₇²-X_{b}-Y₂ (II),

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl; Xₐ and X_{b} are each, independently, a direct bond or a peptidic structure comprising from 1 to about 25 amino acid residues; X₁² is lysine, serine, phenylalanine, glycine or serine; preferably glycine, phenylalanine or serine, most preferably serine; X₂² is serine, tyrosine, valine, alanine or glycine, preferably serine, tyrosine or valine; X₃² is any amino acid residue, but is preferably proline, isoleucine, serine, tryptophan, alanine, valine, tyrosine, or phenylalanine; X₄² is serine, leucine, tyrosine, phenylalanine, asparagine or alanine, preferably serine or leucine; X₅² is leucine, serine, phenylalanine or tyrosine, preferably leucine or serine, most preferably leucine; X₆² is tryptophan; and X₇² is proline.

In one embodiment, the peptide compound comprises the structure: Y₁-Xₐ-Ser-Ser-X₃²-Ser-Leu-Trp-Pro-X_{b}-Y₂; Y₁-Xₐ-Ser-Tyr-X₃²-Ser-Leu-Trp-Pro-X_{b}-Y₂; Y₁-Xₐ-Ser-Val-X₃²-Ser-Leu-Trp-Pro-X_{b}-Y₂; Y₁-Xₐ-Ser-Ser-X₃²-Leu-Leu-Trp-Pro-X_{b}-Y₂; Y₁-Xₐ-Ser-Tyr-X₃²-Leu-Leu-Trp-Pro-X_{b}-Y₂; or Y₁-Xₐ-Ser-Val-X₃²-Leu-Leu-Trp-Pro-X_{b}-Y₂. In another embodiment, the peptide compound comprises the structure: Y₁-Xₐ-Gly-Ser-Pro-Ala-Leu-Trp-Pro-X_{b}-Y₂; Y₁-Xₐ-Leu-Val-Ile-Asp-Leu-Trp-Pro-X_{b}-Y₂; Y₁-Xₐ-Ser-Tyr-Ser-Phe-Leu-Trp-Pro-X_{b}-Y₂; Y₁-Xₐ-Ser-Tyr-Trp-Tyr-Leu-Trp-Pro-X_{b}-Y₂; Y₁-Xₐ-Phe-Tyr-Ala-Ser-Leu-Trp-Pro-X_{b}-Y₂; Y₁-Xₐ-Tyr-Ala-Ala-Ser-Phe-Trp-Pro-X_{b}-Y₂; Y₁-Xₐ-Phe-Ala-Val-Leu-Ser-Trp-Pro-X_{b}-Y₂; Y₁-Xₐ-Gly-Val-Tyr-Leu-Ser-Trp-Pro-X_{b}-Y₂; Y₁-Xₐ-Ser-Ser-Trp-Leu-Ser-Trp-Pro-X_{b}-Y₂; or Y₁-Xₐ-Ser-Gly-Phe-Ser-Tyr-Trp-Pro-X_{b}-Y₂.

In yet another aspect, the invention provides peptide compounds of the formula

Y₁-Xₐ-Ser-Pro-Asp-X₁³-X₂³-Ala-X_{b}-Y₂ (III),

wherein Y₁¹ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl; Xₐ and X_{b} are each, independently, a direct bond or a peptidic structure comprising from 1 to about 25 amino acid residues; X₁³ is Tyr or Leu; and X₂³ is Gln or Leu. In one embodiment, the peptide compound comprises the structure Y₁-Xₐ-Ser-Pro-Asp-Tyr-Gln-Ala-X_{b}-Y₂ or Y₁-Xₐ-Ser-Pro-Asp-Leu-Leu-Ala-X_{b}-Y₂.

In a further aspect, the invention provides peptide compounds of the formula

Y₁ -Xₐ-Ser-Pro-Ala-Leu-Trp-X_{b}-Y₂ (IV),

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl. Xₐ and X_{b} are each, independently, a direct bond or a peptidic structure comprising from 1 to about 25 amino acid residues.

In another aspect, the invention provides peptide compounds of the formula

Y₁-Xₐ-X₁⁵-X₂⁵-Trp-Leu-X₃⁵-Ser-X_{b}-Y₂ (V),

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl. Xₐ and X_{b} are each, independently, a direct bond or a peptidic structure comprising from 1 to about 25 amino acid residues; X₁⁵ is Ser or Pro, preferably Ser; X₂⁵ is Ser or Trp, preferably Ser; and X₃⁵ is Phe or Ala, preferably Ala.

In one embodiment, the peptide compound comprises the structure: Y₁-Xₐ-Ser-Ser-Trp-Leu-Ala-Ser-X_{b}-Y₂; Y₁-Xₐ-Ser-Trp-Trp-Leu-Ala-Ser-X_{b}-Y₂; or Y₁-Xₐ-Pro-Ser-Trp-Leu-Ala-Ser-X_{b}-Y₂.

In yet another aspect, the invention provides peptide compounds of the formula

Y₁-Xₐ-Phe-X₁⁶-X₂⁶-X₃⁶-X₄⁶-X₅⁶-X₆⁶-X_{b}-Y₂ (VI),

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl; Xₐ and X_{b} are each, independently, a direct bond or a peptidic structure comprising from 1 to about 25 amino acid residues; X₁⁶ is Ser or Val, preferably Ser;

X₂⁶ is His, Thr, Gly or Phe, preferably His or Thr; X₃⁶ is Pro, Ile, or Tyr, preferably Pro; X₄⁶ is Ser, Tyr, Ala, Gly or Cys, preferably Ala; X₅⁶ is Trp or Gly, preferably Trp; and X₆⁶ is Leu, Ser, Tyr or Arg, preferably Ser.

In one embodiment the peptide compound comprises the structure: Y₁-Xₐ-Phe-Ser-His-Pro-Ala-Trp-Ser-X_{b}-Y₂; Y₁-Xₐ-Phe-Ser-Thr-Pro-Ala-Trp-Ser-X_{b}-Y₂; Y₁-Xₐ-Phe-Val-His-Pro-Ser-Trp-Leu-X_{b}-Y₂; Y₁-Xₐ-Phe-Ser-His-Pro-Tyr-Trp-Ser-X_{b}-Y₂; Y₁-Xₐ-Phe-Ser-Gly-Pro-Ala-Trp-Ser-X_{b}-Y₂; Y₁-Xₐ-Phe-Ser-Phe-Ile-Ala-Trp-Ser-X_{b}-Y₂; Y₁ -Xₐ-Phe-Ser-Thr-Tyr-Gly-Trp-Tyr-X_{b}-Y₂; or Y₁-Xₐ-Phe-Ser-Thr-Pro-Cys-Gly-Arg-X_{b}-Y₂.

In a further aspect, the invention provides peptide compounds of the formula

Y₁-Xₐ-X₁⁷-X₂⁷-X₃⁷-X₄⁷-X₅⁷-X₆⁷-X₇⁷-X₈⁷-X₉⁷-X_{b}-Y₂ (VII),

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl; Xₐ and X_{b} are each, independently, a direct bond or a peptidic structure comprising from 1 to about 25 amino acid residues; X₁⁷ is Ser, Trp, Phe, Ala or Val, preferably Ser; X₂⁷ is Ser, Leu, Pro, Trp or Val, preferably Ser; X₃⁷ is Ser, Tyr, Val, Met or His, preferably Val, Tyr or Ser, more preferably Tyr or Ser; X₄⁷ is Pro, Ser, Leu, Arg, Tyr, Val or Trp, preferably Pro or Ser, more preferably Pro; X₅⁷ is Phe, Ser, Trp, Arg or Leu, preferably Phe, Trp or Ser; X₆⁷ is Trp, Asn, Met, Asp, Gly, Phe, preferably Trp; X₇⁷ is Ala, Trp, Leu or Ser, preferably Leu; X₈⁷ is Arg, Pro, Phe, Ala, Ser, Tyr, preferably Ala; and X₉⁷ is Pro, Asn, Ser, Trp, Thr, Phe, Ala, preferably Ser or Pro, more preferably Ser.

In one embodiment, the peptide compound comprises the structure: Y₁-Xₐ-Ser-Ser-Ser-Pro-Trp-Trp-Leu-Ala-Ser-Xb-Y2; Y1-Xa-Ser-Ser-Ser-Pro-Phe-Trp-Leu-Ala-Ser-Xb-Y2; Y1-Xa-Ser-Ser-Ser-Pro-Ser-Trp-Leu-Ala-Ser-Xb-Y2; Y1-Xa-Ser-Ser-Tyr-Pro-Trp-Trp-Leu-Ala-Ser-Xb-Y2; Y1-Xa-Ser-Ser-Tyr-Pro-Phe-Trp-Leu-Ala-Ser-Xb-Y2; Y1-Xa-Ser-Ser-Tyr-Pro-Ser-Trp-Leu-Ala-Ser-Xb-Y2; Y1-Xa-Ser-Ser-Val-Pro-Phe-Trp-Ala-Arg-Pro-Xb-Y2; Y₁-Xₐ-Ser-Leu-Val-Pro-Phe-Asn-Trp-Pro-Asn-X_{b}-Y₂; Y₁-Xₐ-Ser-Pro-Tyr-Pro-Ser-Met-Leu-Phe-Ser-X_{b}-Y₂; Y₁-Xₐ-Trp-Trp-Tyr-Pro-Trp-Asp-Leu-Ala-Ser-X_{b}-Y₂; Y₁-Xₐ-Phe-Val-His-Pro-Ser-Trp-Leu-Ala-Ser-X_{b}-Y₂; Y₁-Xₐ-Ala-Ser-Met-Ser-Trp-Trp-Leu-Ala-Ser-X_{b}-Y₂; Y₁-Xₐ-Ser-Ser-Tyr-Ser-Ser-Trp-Leu-Ser-Trp-X_{b}-Y₂; Y₁-Xₐ-Ser-Ser-Ser-Leu-Phe-Trp-Ser-Ala-Thr-X_{b}-Y₂; Y₁-Xₐ-Ser-Ser-Ser-Arg-Ser-Trp-Ala-Ala-Phe-X_{b}-Y₂; Y₁-Xₐ-Ser-Ser-Ser-Tyr-Trp-Gly-Leu-Tyr-Pro-X_{b}-Y₂; Y₁-Xₐ-Ser-Ser-Tyr-Val-Arg-Trp-Leu-Ala-Ala-X_{b}-Y₂; or Y₁-Xₐ-Val-Ser-Ser-Trp-Leu-Phe-Ser-Ala-Ser-X_{b}-Y₂.

In another aspect, the invention provides peptide compounds of the formula

Y₁-Xₐ-Cys-X₁⁸-X₂⁸-X₃⁸-X₄⁸-Gly-X₅⁸-X₆⁸-X₇⁸-Cys-X_{b}-Y₂ (VIII),

wherein Y₁ is hydrogen, alkyl or acyl, preferably hydrogen, linear, branched or cyclic C₁-C₆-alkyl or linear, branched or cyclic C₁-C₆-acyl. Y₂ is —OH, amino or monosubstituted or disubstituted amino, preferably —NR₂, where each R is independently hydrogen or alkyl, preferably linear, branched or cyclic C₁-C₆-alkyl; Xₐ and X_{b} are each, independently, a direct bond or a peptidic structure comprising from 1 to about 25 amino acid residues; X₁⁸ is an aromatic amino acid residue or threonine; X₂⁸ is Gly, Phe, Gln, Arg, Met, Trp; X₃⁸ is Asp or Glu; X₄⁸ is Glu or Asp; X₅⁸ is Tyr or Trp; X₆⁸ is Pro, Trp, Thr, Leu, Phe, Tyr, Met; and X₇⁸ is His, Asp, Ser, Ala, Leu, Met, Trp.

Preferably the peptide compound comprises the structure: Y₁-Xₐ-Cys-Trp-Gly-Asp-Asp-Gly-Trp-Pro-Ala-His-Cys-X_{b}-Y₂; Y₁-Xₐ-Cys-Phe-Phe-Asp-Asp-Gly-Tyr-Trp-Trp-Asp-CYS-X_{b}-Y₂; Y₁-Xₐ-Cys-Tyr-Gln-Asp-Asp-Gly-Tyr-Thr-Val-Ser-Cys-X_{b}-Y₂; Y₁-Xₐ-Cys-Tyr-Met-Glu-Asp-Gly-Tyr-Leu-Trp-Ala-Cys-X_{b}-Y₂; Y₁-Xₐ-Cys-Phe-Phe-Glu-Asp-Gly-Tyr-Phe-His-Ala-Cys-X_{b}-Y₂; Y₁-Xₐ-Cys-Tyr-Gly-Asp-Asp-Gly-Trp-Phe-Met-Leu-Cys-X_{b}-Y₂; Y₁-Xₐ-Cys-Tyr-Met-Asp-Glu-Gly-Trp-Tyr-Tyr-Ser-Cys-X_{b}-Y₂; Y₁-Xₐ-Cys-Trp-Gln-Glu-Asp-Gly-Trp-Leu-Tyr-Leu-Cys-X_{b}-Y₂; Y₁-Xₐ-Cys-Trp-Arg-Glu-Asp-Gly-Tyr-Trp-Trp-Trp-Cys-X_{b}-Y₂; Y₁-Xₐ-Cys-Trp-Gln-Asp-Asp-Gly-Trp-Tyr-Tyr-Met-Cys-X_{b}-Y₂; Y₁-Xₐ-Cys-Tyr-Trp-Asp-Asp-Gly-Trp-Met-Leu-Glu-Cys-X_{b}-Y₂; Y₁-Xₐ-Cys-Trp-Arg-Glu-Asp-Gly-Tyr-Trp-Trp-Trp-Cys-X_{b}-Y₂; or Y₁-Xₐ-Cys-Thr-Trp-Asp-Asp-Gly-Trp-Met-Phe-Leu-Cys-X_{b}-Y₂.

Preferably, at least one of Xa and Xb comprises an amino acid sequence which facilitates transport of the peptide into mammalian cells and, preferably, into the nuclei of mammalian cells.

The peptide compounds of the invention also include peptides having sequences which are derived from the sequences of formulas I-VIII by replacement of two or more amino acid residues with a peptidomimetic structure. Preferred peptidomimetic structures are one-, two- and three-amino acid residue analogues which present the side chains presented by the amino acid residues they replace in formulas I-VIII.

In one embodiment, Xₐ and/or X_{b} comprise an amino acid sequence which facilitates the transport of the peptide compound across cell and/or nuclear membranes. Such peptides are able to enter cells and/or the nucleus and interact with cytosolic or nuclear androgen receptor. Suitable amino acid sequences which enable the peptide compounds of the invention to cross cell membranes are known in the art and include the Kaposi FGF signal sequence (U.S. Pat. No. 5,807,746; U.S. Pat. No. 5,962,415); sequences derived from the HIV TAT protein (U.S. Pat. No. 5,804,604; U.S. Pat. No. 5,670,617 and U.S. Pat. No. 5,747,641); the antennapedia homeodomain (U.S. Pat. No. 5,888,762; U.S. Pat. No. 6,080,724) and truncated variants (PCT Application No. WO 00/29427); sequences derived from gelsolin (U.S. Pat. No. 5,846,743; U.S. Pat. No. 5,783,662) and other sequences, as are described in PCT WO 99/29721. Each of the foregoing references is incorporated herein by reference in its entirety. Preferably, one of Xₐ and X_{b} comprises one of the cell permeable amino acid sequences disclosed in these references.

As used herein, the terms “peptide compound” and “peptidic structure” are intended to include peptides comprised of naturally-occurring amino acids, as well as peptide derivatives, peptide analogues and peptide mimetics of the naturally-occurring amino acid structures. The terms “peptide analogue”, “peptide derivative” and “peptidomimetic” as used herein are intended to include molecules which mimic the chemical structure of a peptide and retain the functional properties of the peptide. Approaches to designing peptide analogues, derivatives and mimetics are known in the art. For example, see Farmer, P.S. in Drug Design (E. J. Ariens, ed.) Academic Press, New York, 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270.

As used herein, a “derivative” of a compound X (e.g., a peptide or amino acid) refers to a form of X in which one or more reaction groups on the compound have been derivatized with a modifying (derivative) group. Examples of peptide derivatives include peptides in which an amino acid side chain, the peptide backbone, or the amino- or carboxy-terminus has been derivatized (e.g., peptidic compounds with methylated amide linkages).

An “analogue” of a reference amino acid, as the term is used herein, is an α- or β-amino acid having a side chain which is (a) the same as the side chain of the reference amino acid (when the analogue is a β-amino acid residue, a peptoid, or the D-amino acid enantiomer of the reference acid); (b) is an isomer of the side chain of the reference amino acid; (c) is a homologue of the side chain of the reference amino acid; (d) results from replacement of a methylene group in the side chain of the reference amino acid with a heteroatom or group selected from NH, O and S; (e) results from a simple substitution on the side chain of the reference amino acid or any of the preceding (a) to (c); and/or (f) results from a conservative substitution (discussed infra). Analogues of a reference amino acid further include the reference amino acid or any of (a)-(e) above in which the α-nitrogen atom is substituted by a lower alkyl group, preferably a methyl group. A “homologue” of the given amino acid is an α- or β-amino acid having a side chain which differs from the side chain of the given amino acid by the addition or deletion of from 1 to 4 methylene groups. A “simple substitution” of an amino acid side chain results from the substitution of a hydrogen atom in the side chain of the given amino acid with a small substituent, such as a lower alkyl group, preferably a methyl group; a halogen atom, preferably a fluorine, chlorine, bromine or iodine atom; or hydroxy.

Peptide mimetics that are structurally similar to therapeutically useful peptides may be used to produce an equivalent therapeutic or prophylactic effect. The term mimetic, and in particular, peptidomimetic, is intended to include isosteres. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) well known to those skilled in the art. Generally, peptidomimetics are structurally similar to a paradigm peptide (i.e., a peptide that has a biological or pharmacological activity), but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2—); Spatola, A. F. et al. (1986) Life Sci. 38:1243-1249 (—CH2—S); Hann, M. M. (1982) J. Chem. Soc. Perkin Trans. I. 307-314 (—CH—CH—, cis and trans); Almquist, R. G. et al. (1980) J. Med. Chem. 23:1392-1398 (—COCH2—); Jennings-White, C. et al. (1982) Tetrahedron Lett. 23:2533 (—COCH2—); Szelke, M. et al. European Appln. EP 45665 (1982) CA: 97:39405 (1982)(—CH(OH)CH2—); Holladay, M. W. et al. (1983) Tetrahedron Lett. 24:4401-4404 (—C(OH)CH2—); and Hruby, V. J. (1982) Life Sci. 31:189-199 (—CH2—S—); each of which is incorporated herein by reference. A particularly preferred non-peptide linkage is —CH2NH—.

Other examples of isosteres include peptides substituted with one or more benzodiazepine molecules (see e.g., James, G. L. et al. (1993) Science 260:1937-1942). Other possible modifications include an N-alkyl (or aryl) substitution (ψ{CONR}), backbone crosslinking to construct lactams and other cyclic structures, substitution of all D-amino acids for all L-amino acids within the compound (“inverso” compounds) or retro-inverso amino acid incorporation (ψ{NHCO}). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide. See Goodman et al. “Perspectives in Peptide Chemistry” pp. 283-294 (1981). See also U.S. Pat. No. 4,522,752 by Sisto for further description of “retro-inverso” peptides. Other derivatives include C-terminal hydroxymethyl derivatives, O-modified derivatives (e.g., C-terminal hydroxymethyl benzyl ether) and N-terminally modified derivatives including substituted amides such as alkylamides and hydrazides.

Such peptide mimetics may have significant advantages over peptide embodiments, including, for example: more economical production, greater chemical stability, enhanced pharmacological properties (e.g., half-life, absorption, potency, efficacy, and the like), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivitization (e.g., labeling) of peptidomimetics should not substantially interfere with the desired biological or pharmacological activity of the peptidomimetic.

Systematic substitution of one or more amino acids of an amino acid sequence with a D-amino acid of the same type (e.g., D-lysine in place of L-lysine) may be used to generate more stable peptides. In addition, constrained peptides may be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The term “conservative substitution”, as used herein, includes the replacement of one amino acid residue by another residue having similar side chain properties. As is known in the art, the twenty naturally amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group.

One subset of preferred compounds of formula I have the amino acid sequences set forth below (where lower case characters represent D-amino acids).

| | |
|---|---|
| KVESSSVFSKPASVTVASDA | (SEQ ID NO:1) |
| PAAIPPSLTDYSVPFHHTPVSSMSSDL | (SEQ ID NO:2) |
| LTIESSSDEEEDPPAKRKAIF | (SEQ ID NO:3) |
| KPASVTVASDASKKIDVIDL | (SEQ ID NO:4) |
| LTIESSSDEE | (SEQ ID NO:5) |
| FMSETQSSPTK | (SEQ ID NO:6) |
| SVPFHHTPVSSMSSDL | (SEQ ID NO:7) |
| SVPFHHTPVS | (SEQ ID NO:8) |
| TPVSSMSSDL | (SEQ ID NO:9) |
| LTIESSSDEE | (SEQ ID NO:10) |
| ATIESSSDEE | (SEQ ID NO:11) |
| LAIESSSDEE | (SEQ ID NO:12) |
| LTAESSSDEE | (SEQ ID NO:13) |
| LTIASSSDEE | (SEQ ID NO:14) |
| LTIEASSDEE | (SEQ ID NO:15) |
| LTIESASDEE | (SEQ ID NO:16) |
| LTIESSSAEE | (SEQ ID NO:17) |
| LTIESSSDAE | (SEQ ID NO:18) |
| LTIESSSDEA | (SEQ ID NO:19) |
| LTIESSSDE | (SEQ ID NO:20) |
| TIESSSDEE | (SEQ ID NO:21) |
| TIESSSDE | (SEQ ID NO:22) |
| TIESSSD | (SEQ ID NO:23) |
| LTIESSSDEE | (SEQ ID NO:24) |
| IES(B-Ala)SD | (SEQ ID NO:25) |
| IESASD | (SEQ ID NO:26) |
| IASASD | (SEQ ID NO:27) |
| IESFSD | (SEQ ID NO:28) |
| IESLSD | (SEQ ID NO:29) |
| IESVSD | (SEQ ID NO:30) |
| DSSSEI | (SEQ ID NO:31) |
| Iesssd | (SEQ ID NO:32) |
| Dsssei | (SEQ ID NO:33) |
| TIESSS | (SEQ ID NO:34) |
| IESSSDEE | (SEQ ID NO:35) |
| IESSSD | (SEQ ID NO:36) |
| ESSSDE | (SEQ ID NO:37) |
| SSSDEE | (SEQ ID NO:38) |
| SSSDE | (SEQ ID NO:39) |
| IESSSDE | (SEQ ID NO:40) |
| YGRKKRRQRRRGGGGLTIESSSDEE | (SEQ ID NO:41) |

One subset of preferred compounds of formula II have the following amino acid sequences: Ser-Met-Phe-Gly-Ser-Pro-Ala-Leu-Trp-Pro-Leu-Arg (SEQ ID NO:56); Leu-Val-Ile-Asp-Leu-Trp-Pro-Ala-Phe-Val-Arg (SEQ ID NO:57); Ser-Tyr-Ser-Phe-Leu-Trp-Pro-Ile-Val-Phe-Lys (SEQ ID NO:58); Glu-Leu-Phe-Ser-Tyr-Trp-Pro-Trp-Pro-Phe-Tyr-Arg (SEQ ID NO:59); Phe-Tyr-Ala-Ser-Leu-Trp-Pro-His-Leu-Tyr-Val (SEQ ID NO:60); Val-Gly-Thr-Gly-Ala-Ser-Ser-Tyr-Ala-Ala-Ser-Phe-Trp-Pro-Trp-Met-Thr-Tyr-Tyr-Trp (SEQ ID NO:61); Phe-Ala-Val-Leu-Ser-Trp-Pro-Leu-Tyr-Glu-Tyr (SEQ ID NO:62); Gly-Val-Tyr-Leu-Ser-Trp-Pro-Gly-Ser-Met-Ala (SEQ ID NO:63); Ser-Ser-Tyr-Ser-Ser-Trp-Leu-Ser-Trp-Pro-Arg (SEQ ID NO:64); or Ser-Gly-Phe-Ser-Tyr-Trp-Pro-Phe-Phe-Phe-Val (SEQ ID NO:65).

One subset of preferred compounds of formula III have the following amino acid sequences: Arg-Met-Val-Ser-Pro-Asp-Tyr-Gln-Ala-Thr-Ser-Pro (SEQ ID NO:66) or Leu-Ser-Phe-Ser-Pro-Asp-Leu-Leu-Ala-Leu-Arg-Gly-Met (SEQ ID NO:67).

One subset of preferred compounds of formula IV have the following amino acid sequences: Ser-Met-Phe-Gly-Ser-Pro-Ala-Leu-Trp-Pro-Leu-Arg (SEQ ID NO:68) or Val-Ser-Pro-Ala-Leu-Trp-Ser-Ser-Leu-Arg-Gly (SEQ ID NO:69).

One subset of preferred compounds of formula V have the following amino acid sequences: Gly Phe Val Ser Ser Trp Leu Phe Ser Ala Ser (SEQ ID NO:70); Ala Ser Met Ser Tip Trp Leu Ala Ser Ser Pro (SEQ ID NO:71); or Phe Val His Pro Ser Trp Leu Ala Ser Phe Leu (SEQ ID NO:72).

One subset of preferred compounds of formula VI have the following amino acid sequences: Phe Val His Pro Ser Trp Leu Ala Ser Phe Leu (SEQ ID NO:73); Phe Ser His Pro Tyr Trp Ser Tyr Leu Phe Ser (SEQ ID NO:74); Phe Ser Gly Pro Ala Trp Ser Leu His Lys His (SEQ ID NO:75); Phe Ser Phe Ile Ala Trp Ser Pro Ala Met Leu (SEQ ID NO:76); Phe Ser Thr Tyr Gly Trp Tyr Ser Pro Phe His (SEQ ID NO:77); or Ile Pro Phe Ser Thr Pro Cys Gly Arg Trp Cys (SEQ ID NO:78).

One subset of preferred compounds of formula VII have the following amino acid sequences: Ser-Ser-Val-Pro-Phe-Trp-Ala-Arg-Pro-Leu-Val (SEQ ID NO:79); Ser-Leu-Ser-Ser-Leu-Val-Pro-Phe-Asn-Trp-Pro-Asn-Leu-Phe-Ser-Trp-Arg-Tyr-Ser-Trp (SEQ ID NO:80); Leu-Gly-Ser-Pro-Tyr-Pro-Ser-Met-Leu-Phe-Ser-Asp-His (SEQ ID NO:81); Phe-Trp-Trp-Tyr-Pro-Trp-Asp-Leu-Ala-Ser-Tyr (SEQ ID NO: 82); Phe-Val-His-Pro-Ser-Trp-Leu-Ala-Ser-Phe-Leu (SEQ ID NO:83); Ala-Ser-Met-Ser-Trp-Trp-Leu-Ala-Ser-Ser-Pro (SEQ ID NO:84); Ser-Ser-Tyr-Ser-Ser-Trp-Leu-Ser-Trp-Pro-Arg (SEQ ID NO:85); Ser-Ser-Ser-Leu-Phe-Trp-Ser-Ala-Thr-Ser-Arg (SEQ ID NO:86); Ser-Ser-Ser-Arg-Ser-Trp-Ala-Ala-Phe-Glu-His (SEQ ID NO:87); Ser-Ser-Ser-Tyr-Trp-Gly-Leu-Tyr-Pro-Ser-Leu-Ser-Leu (SEQ ID NO:88); Ser-Ser-Tyr-Val-Arg-Trp-Leu-Ala-Ala-Ala-Gln (SEQ ID NO:89); Gly-Phe-Val-Ser-Ser-Trp-Leu-Phe-Ser-Ala-Ser (SEQ ID NO:90); or Ser-His-Gly-Trp-Phe-Trp-Ser-Ser-Ser-Gln-Gly (SEQ ID NO:91).

II. Preparation of the Peptide Compounds of the Invention

Those of skill in the art can, without undue experimentation, produce peptides corresponding to particular peptide sequences. Such peptides may be produced in prokaryotic or eukaryotic host cells by expression of polynucleotides encoding the particular peptide sequence. Alternatively, such peptides may be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken, I. M. (1981) CRC Crit. Rev. Biochem. 11:255; Kaiser et al. (1989) Science 243:187; Merrifield, B. (1986) Science 232:342; Kent, S. B. H. (1988) Ann. Rev. Biochem. 57:957; and Offord, R. E. (1980) Semisynthetic Proteins, Wiley Publishing, which are incorporated herein in their entirety by reference).

Peptides can be produced, e.g., by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, may be used when synthesizing the peptide compounds of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others.

The peptide compounds of the present invention may also be prepared by any suitable method for chemical peptide synthesis, including solution-phase and solid-phase chemical synthesis. Preferably, the peptides are synthesized on a solid support. Methods for chemically synthesizing peptides are well known in the art (see, e.g., Bodansky, M. Principles of Peptide Synthesis, Springer Verlag, Berlin (1993) and Grant, G. A (ed.). Synthetic Peptides: A User's Guide, W. H. Freeman and Company, New York (1992). Automated peptide synthesizers useful to make the peptide compounds of this invention are commercially available.

III. Modifying Groups

In certain embodiments, the peptide compounds of the invention are coupled directly or indirectly to a modifying group. The term “modifying group” is intended to include structures that are directly attached to the amino acid peptidic structure (e.g., by covalent coupling), as well as those that are indirectly attached to the peptidic structure (e.g., by a stable non-covalent association). For example, the modifying group can be coupled to the amino-terminus or carboxy-terminus of the peptide compounds of the invention. Alternatively, the modifying group can be coupled to a side chain of at least one amino acid residue of a peptide compound of the invention (e.g., through the epsilon amino group of a lysyl residue(s), through the carboxyl group of an aspartic acid residue(s) or a glutamic acid residue(s), through a hydroxy group of a tyrosyl residue(s), a serine residue(s) or a threonine residue(s) or other suitable reactive group on an amino acid side chain). Modifying groups covalently coupled to the amino acid peptidic structure can be attached by means and using methods well known in the art for linking chemical structures, including, for example, amide, alkylamino, carbamate, urea or ester bonds.

In a preferred embodiment, the modifying group(s) comprises an alkyl group. The term “alkyl”, as used herein, refers to a straight or branched chain hydrocarbon group having from about 1 to about 10 carbon atoms. Exemplary alkyl groups include methyl, ethyl, dimethyl, diethyl, n-propyl, isopropyl, n-butyl, isobutyl, sec-butyl, tert-butyl, n-pentyl, and n-hexyl. A “lower alkyl group”, as the term is used herein, is a linear, branched or cyclic C₁-C₆-alkyl. Preferred lower alkyl groups include linear or branched C₄-alkyl and C₃-alkyl; ethyl and methyl groups. An alkyl group may be unsubstituted, or may be substituted at one or more positions, with, e.g., halogens, alkyls, cycloalkyls, alkenyls, alkynyls, aryls, heterocycles, hydroxyls, aminos, nitros, thiols, amines, imines, amides, phosphonates, phosphines, carbonyls, carboxyls, silyls, ethers, thioethers, sulfonyls, selenoethers, ketones, aldehydes, esters, —CF₃, —CN, or the like. Preferred alkyls are methyls, ethyls, dimethyls, diethyls, n-propyls, or isopropyls.

In another embodiment, one modifying group, e.g., an alkyl group, is coupled to another modifying group. In yet another embodiment, a D-amino acid in a peptide compound of the invention is modified with two modifying groups.

The amino- and/or carboxy-terminus of the peptide compounds of the invention can be unmodified hydrogen. Alternatively, the amino- and/or carboxy-terminus of the peptide compound can be modified with a derivative group. Amino-derivative groups which can be present at the N-terminus of a peptide compound include acetyl, aryl, alkyl, aralkyl, acyl, epoxysuccinyl and cholesteryl groups. Carboxy-derivative groups which can be present at the C-terminus of a peptide compound include alcohol, aldehyde, amine, epoxysuccinate, acid halide, carbonyl, halomethane, and diazomethane groups.

VI. Methods for Treating Androgen-Associated Disorders

The present invention also provides a method of treating an androgen associated disorder in a subject. The method includes administering to the subject a therapeutically effective amount of a peptide compound of the invention. Preferably, the peptide compound is cell permeable or includes an amino acid sequence which facilitates the passage of the peptide compound across a cell membrane.

As used herein, an “androgen-associated disorder” includes a disease, disorder, or condition, which proceeds, directly or indirectly, via androgen receptor-induced gene transcription. Androgen associated disorders include benign prostatic hypertrophy, male pattern baldness, acne, idiopathic hirsutism, and Stein-Leventhal syndrome. Androgen associated disorders further include cancers whose growth is promoted by Androgens. Examples of Androgen promoted cancers include prostate cancer (Mendelson (2000) Prog Drug Res 55:213-33), ovarian cancer (Ilekis, et al. (1997) Gynecol Oncol. 66(2):250-4), bladder cancer (Zhuang, et al. (1997) Histopathology 30(6):556-62), colon cancer (Catalano, et al. (2000) Int. J. Cancer 86(3):325-30), liver cancer (Cui (1995) Chung Hua Chung Liu Tsa Chih 17(4):304-6), endometrial cancer (Hackenberg, et al. (1994) Int. J. Cancer 57(1):117-22), pancreatic cancer (Greenway (2000) Drugs Aging 17(3):161-3), lung cancer (Maasberg, et al. (1989) Int. J. Cancer 43(4):685-91), esophageal cancer (Yamashita, et al. (1989) Jpn J Surg 19(2):195-202), cancer of the larynx (Marugo, et al. (1987) J Endocrinol Invest 10(5):465-70), and breast cancer. Other androgen-associated disorders include androgen insensitivity syndromes, infertility, endometrial cancer, and X-linked spinal bulbar muscular atrophy (SMBA). Examples of partial androgen insensitivity syndromes include incomplete testicular feminization, Reifenstein syndrome, Lubs syndrome, Gilbert-Dreifus syndrome, and Rosewater syndrome.

As used herein, the term “subject” includes warm-blooded animals, preferably mammals, including humans. In a preferred embodiment, the subject is a primate. In an even more preferred embodiment, the primate is a human.

As used herein, the term “administering” to a subject includes dispensing, delivering or applying a peptide compound of the invention e.g., a peptide compound in a pharmaceutical formulation, to a subject by any suitable route for delivery of the composition to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route.

As used herein, the term “therapeutically effective amount” includes an amount effective, at dosages and for periods of time necessary, to achieve the desired result, e.g., sufficient to treat an androgen associated disorder in a subject. An effective amount of a peptide compound of the invention, as defined herein may vary according to factors such as the disease state, age, and weight of the subject, and the ability of the peptide compound to elicit a desired response in the subject. Dosage regimens may be adjusted to provide the optimum therapeutic response. An effective amount is also one in which any toxic or detrimental effects (e.g., side effects) of the peptide compound are outweighed by the therapeutically beneficial effects.

V. Pharmaceutical Compositions

Another aspect of the invention pertains to pharmaceutical compositions of the peptide compounds of the invention. The pharmaceutical compositions of the invention typically comprise a peptide compound of the invention and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, monostearate salts and gelatin. Moreover, the compounds can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Depending on the route of administration, the compound may be coated in a material to protect it from the action of enzymes, acids and other natural conditions which may inactivate the agent. For example, the compound can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluoro-phosphate (DEP) and trasylol. Liposomes include water-in-oil-in-water emulsions as well as conventional liposomes (Strej an, et al., (1984) J. Neuroimmunol 7:27). Dispersions can also be prepared in glycerol, liquid polyethylene glycols, and mixtures thereof and in oils. Under ordinary conditions of storage and use, these preparations may contain a preservative to prevent the growth of microorganisms.

The active agent in the composition (i.e., a peptide compound of the invention) preferably is formulated in the composition in a therapeutically effective amount. A “therapeutically effective amount” refers to an amount effective, at dosages and for periods of time necessary, to achieve the desired therapeutic result, such as modulation of androgen receptor activity to thereby influence the therapeutic course of a particular disease state. A therapeutically effective amount of an active agent may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. In another embodiment, the active agent is formulated in the composition in a prophylactically effective amount. A “prophylactically effective amount” refers to an amount effective, at dosages and for periods of time necessary, to achieve the desired prophylactic result, for example, modulation of androgen receptor activity for prophylactic purposes. Typically, since a prophylactic dose is used in subjects prior to or at an earlier stage of disease, the prophylactically effective amount will be less than the therapeutically effective amount.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

A peptide compound of the invention can be formulated into a pharmaceutical composition wherein the compound is the only active agent therein. Alternatively, the pharmaceutical composition can contain additional active agents. For example, two or more peptide compounds of the invention may be used in combination. Moreover, a peptide compound of the invention can be combined with one or more other agents that have modulatory effects on androgen receptor activity.

VI. Screening Assays

The invention also provides a method of assessing the ability of a compound to bind to the androgen receptor or an androgen receptor functional domain, such as the DNA binding domain. The method includes (1) contacting the androgen receptor with a peptide of formulas I-VIII, thereby forming a peptide compound-androgen receptor complex; contacting the peptide compound-androgen receptor complex with a test compound; and determining if the peptide compound dissociates from the peptide compound-androgen receptor complex in the presence of the test compound, thereby identifying a compound that binds to the androgen receptor. If the peptide dissociates in the presence of the test compound, then the test compound binds to the androgen receptor and displaces the peptide. This method can utilize the intact androgen receptor or a fragment thereof, such as a functional domain of the intact receptor. The androgen receptor fragment can also be present as a fusion protein, such as a glutathione-S-transferase fusion protein. Preferably, the androgen receptor DNA binding domain is used.

The foregoing method can be performed using methods which are known in the art. Test compounds can be assessed singly or in libraries. In one embodiment, the androgen receptor or androgen receptor functional domain is linked to a solid support, for example, in a chromatography column, and treated with a peptide of formulas I-VIII. The test compound or compounds are then added to the column and the column effluent is analyzed for the presence of the test compound and the peptide. Suitable methods for analyzing the effluent are known in the art and include gas or liquid chromatography in combination with mass spectroscopy.

Gel shift assays can be used to confirm the binding affinity and specificity between androgen receptor protein (AR) and androgen response elements (ARE). In a gel shift assay, ARE that is bound to AR will migrate more slow than unbound ARE. CAT assays can be used to confirm that AR has bound and activated transcription at the ARE. In CAT assays, the ARE is linked upstream to the CAT reporter gene so that AR causes the activation of the CAT reporter gene. Gel shift assays and CAT assays can be used to identify peptides that bind to AR and that inhibit AR gene activation. A luciferase reporter assay, e.g., the Dual-Luciferase™ Reporter Assay (available through Promega and described in Sherf, B. A. et al. (1996) Promega Notes Magazine 57:02); and the secreted alkaline phosphatase (SEAP) assay described in, for example, Berger, J. et al. Gene 66: 1-10, the contents of which are incorporated herein by reference, may also be used.

In another embodiment, the invention relates to a method of assessing the ability of a test compound to inhibit androgen receptor-DNA binding. The method includes (1) contacting a peptide comprising a DNA-binding domain of the androgen receptor with the test compound and a DNA sequence comprising an androgen recognition element; and (2) determining binding, e.g., the extent of binding, of the DNA sequence to the peptide. The extent of binding determined in step (2) may be determined quantitatively and may further be compared to the extent of binding of the DNA sequence to the peptide in the absence of the test compound. The DNA sequence can be a single-stranded or double-stranded polynucleotide sequence.

The extent of binding of the ligand to the polypeptide may be determined using a method that ncludes the use of a peptide attached to a first label and a DNA sequence attached to a second label. In a preferred embodiment, one of the first and second labels is a magnetic bead and the other is an electroactive transition metal-containing moiety. The labeled peptide, the labeled DNA and the test compound are combined to form an assay mixture which is contacted with a magnetic electrode under conditions such that in the absence of test compound the peptide binds the DNA to form a complex and substantially only transition metal moiety within the complex (and not transition metal moiety attached to free (unbound) peptide or DNA) is excited at the electrode and fluoresces. Thus, the fluorescence is a measure of the amount of complex formed.

In one embodiment, the assay mixture further comprises a polynucleotide which is not an androgen responsive element to eliminate non-specific binding of the peptide to the labeled DNA.

In a preferred embodiment, the peptide is a fusion protein comprising the androgen receptor DNA binding domain and a second protein, such as glutathione-S-transferase. The transition metal moiety can, for example, be attached via an antibody to the second protein. The transition metal moiety metal can be any electroactive metal complex which can be electroactivated to a fluorescent excited state and is, preferably, a complex of Ru(II), such as [Ru(bipyridine)₃]²⁺. An example of a suitable instrument for performing an assay of this type is the IGEN M8 Electrochemiluminescence Detection Unit (IGEN International, Inc. Gaithersburg, Md.).

Examples of androgen responsive elements (ARE) that may be used in the screening assays of the invention are summarized in the Table below.

| ARE Sequences | | Genes |
|---|---|---|
| AGTACGtgaTGTTCT | (SEQ ID NO:42) | C3 |
| GAAACAgccTGTTCT | (SEQ ID NO:43) | SLP |
| AGCACTtgcTGTTCT | (SEQ ID NO:44) | PSA |
| ATAGCAtctTGTTCT | (SEQ ID NO:45) | Probasin (ARE1) |
| AGTCCCactTGTTCT | (SEQ ID NO:46) | ODC |
| AGTACTtgtTGTTCT | (SEQ ID NO:47) | GUS (intron 9) |
| AGCTCAgctTGTACT | (SEQ ID NO:48) | Factor IX |
| AGAACAaccTGTTGA | (SEQ ID NO:49) | MEP-24 |
| TGAAGTtccTGTTCT | (SEQ ID NO:50) | MVDP |
| GTAAAGTACTCCAAGAA | (SEQ ID NO:51) | Probasin (ARE2) |
| GGAACAGCAAGTGCT | (SEQ ID NO:52) | KLK2 |

ARE sequences having the consensus sequence GGAATACAnnnTGTTCT (SEQ ID NO:53) may also be used.

The invention is further illustrated by the following examples, which should not be construed as further limiting. The contents of all references, pending patent applications and published patents, cited throughout this application, as well as the Sequence Listing are hereby expressly incorporated by reference.

### EXAMPLES

Synthetic Peptides

A series of peptides based on amino acids 423-548 of the androgen receptor-interacting protein ARIP3 (Moilanen et al. (1999) J. Biol. Chem. 274: 3700-3704) and sequence variants thereof were synthesized using standard methods of peptide synthesis. These peptides were assessed for the ability to inhibit binding of the androgen receptor to an androgen responsive element using the method described below.

ARE Binding Assay

Anti-glutathione S-transferase monoclonal antibody was purchased from Upstate Biotechnology, Inc., Lake Placid, N.Y. (catalogue #05-311). Prior to ruthenium labeling, 1 mg/ml antibody was dialyzed against phosphate-buffered saline, pH 7.2, for 24 hours at 4° C. A 0.5 mg of antibody was incubated with 18.8 μg Origen TAG-succinimidyl ester (IGEN, International, Inc. Gaithersburg, Md., catalogue # 110034) for 1 hour at room temperature in the dark. The reaction was stopped with 20 μl 2M glycine for 10 minutes in the dark. Free label was removed with a NAP-5 column. Protein concentration was determined using the micro-BCA assay (catalogue #23235ZZ) from Pierce, Rockford, Ill.

Oligonucleotides were purchased from Integrated DNA Technologies, Inc., Coralville, Iowa. One nmole of each complementary oligonucleotide, Biotin-5′-AGTCTGGTACAGGGTGTTCTTTTTA-3′ (SEQ ID NO:54) and 5′-TAAAAAGAACACCCTGTACCAGACT-3′ (SEQ ID NO:55) was added to 10 μl water and heated to 95° C. for 2 minutes. NaCl was added to a final concentration of 50 mM and the oligonucleotides were allowed to anneal at room temperature. After 1 hour, the annealed oligonucleotides were resuspended in 1 ml water.

The glutathione S-transferase/human androgen receptor DNA binding domain (amino acids 505-635) (GST-DBD) was obtained from Dr. Steven Balk. Synthesis of similar androgen receptor fusion proteins has been previously described (Sun, Z., Pan, J. and Balk, S. P. (1997) Nucleic Acids Research 25:3318-3325). The competition binding assay was performed in phosphate-buffered saline, pH 7.2, containing 100 ng/ml poly dI/dC (Sigma-Aldrich Co., St. Louis, Mo., catalogue #P4929) in a final volume of 0.2 ml by incubating 3 ng oligonucleotide, 200 ng GST-DBD, 30 ng antibody and the desired concentration of test compound. After 1.5 hours in the dark at room temperature, 12.5 μg streptavidin-coated M-280 Dynabeads (IGEN International, Inc. Gaithersburg, Md., catalogue # 402-175-02) were added and incubated while shaking for 30 minutes in the dark. Plates were analyzed on an IGEN M8 Electrochemiluminescence Detection Unit (IGEN, International, Inc. Gaithersburg, Md.).

Results of the ARE Binding Assay

The results of the ARE binding assay are shown in the table below. The results are expressed as an IC₅₀ for each peptide. In cases where more than one assay was performed, the indicated IC₅₀ is an average and the number of replications is indicated in parentheses.

| Cmpd No. | | IC₅₀(μM) |
|---|---|---|
| 1 | Ac-KVESSSVFSKPASVTVASDA-NH₂ | 34 (2) |
| (SEQ ID NO:1) | | |
| 2 | Ac-KPASVTVASDASKKIDVIDL-NH₂ | >50 |
| (SEQ ID NO:4) | | |
| 3 | Ac-LTIESSSDEEEDPPAKRKAIF-NH₂ | 22 (2) |
| (SEQ ID NO:3) | | |
| 4 | Ac-EDPPAKRKAIFMSETQSSPTK-NH₂ | >50 |
| (SEQ ID NO:92) | | |
| 5 | Ac-FMSETQSSPTKGVLMYQPSSV-NH₂ | 15 (2) |
| (SEQ ID NO:93) | | |
| 6 | Ac-KGVLMYQSSVRVPSVTSVDP-NH₂ | >50 |
| (SEQ ID NO:94) | | |
| 7 | Ac-VRVPSVTSVDPAAIPPSLTDY-NH₂ | >50 |
| (SEQ ID NO:95) | | |
| 8 | Ac-PAAIPPSLTDYSVPFHHTPVSSMSSDL-NH₂ | 28 (2) |
| (SEQ ID NO:2) | | |
| 9 | Ac-KVESSSVFS-NH₂ | >50 |
| (SEQ ID NO:96) | | |
| 10 | Ac-LTIESSSDEE-NH₂ | 4.7 (6) |
| (SEQ ID NO:5) | | |
| 11 | Ac-FMSETQSSPTK-NH₂ | 5.6 (2) |
| (SEQ ID NO:6) | | |
| 12 | Ac-SVPFHHTPVSSMSSDL-NH₂ | 8.2 (2) |
| (SEQ ID NO:7) | | |
| 13 | Ac-SVPFHHTPVS-NH₂ | 27 |
| (SEQ ID NO:8) | | |
| 14 | Ac-TPVSSMSSDL-NH₂ | 7.4 (2) |
| (SEQ ID NO:9) | | |
| 15 | H-LTIESSSDEE-NH₂ | 25 |
| (SEQ ID NO:10) | | |
| 16 | Ac-ATIESSSDEE-NH₂ | 25 |
| (SEQ ID NO:11) | | |
| 17 | Ac-LAIESSSDEE-NH₂ | 25 |
| (SEQ ID NO:12) | | |
| 18 | Ac-LTAESSSDEE-NH₂ | 25 |
| (SEQ ID NO:13) | | |
| 19 | Ac-LTIASSSDEE-NH₂ | 6.6 (2) |
| (SEQ ID NO:14) | | |
| 20 | Ac-LTIEASSDEE-NH₂ | 18 |
| (SEQ ID NO:15) | | |
| 21 | Ac-LTIESASDEE-NH₂ | 1.5 (2) |
| (SEQ ID NO:16) | | |
| 22 | Ac-LTIESSSAEE-NH₂ | 20 |
| (SEQ ID NO:17) | | |
| 23 | Ac-LTIESSSDAE-NH₂ | 14 |
| (SEQ ID NO:18) | | |
| 24 | Ac-LTIESSSDEA-NH₂ | 2.6 (3) |
| (SEQ ID NO:19) | | |
| 25 | Ac-LTIESSSDE-NH₂ | 30 (2) |
| (SEQ ID NO:20) | | |
| 26 | Ac-LTIESS-NH₂ | >50 |
| (SEQ ID NO:97) | | |
| 27 | Ac-TIESSSDEE-NH₂ | 22 |
| (SEQ ID NO:21) | | |
| 28 | Ac-TIESSSDE-NH₂ | 22 |
| (SEQ ID NO:22) | | |
| 29 | Ac-TIESSSD-NH₂ | 40 |
| (SEQ ID NO:23) | | |
| 30 | Ac-LTIESSSDEE-OH | 7.4 |
| (SEQ ID NO:24) | | |
| 31 | Ac-IES(B-Ala)SD-NH₂ | 3.3 (2) |
| (SEQ ID NO:25) | | |
| 32 | Ac-IESASD-NH₂ | 20 (2) |
| (SEQ ID NO:26) | | |
| 33 | Ac-IASASD-NH₂ | 20 (2) |
| (SEQ ID NO:27) | | |
| 34 | Ac-IESFSD-NH₂ | 63 (2) |
| (SEQ ID NO:28) | | |
| 35 | Ac-IESLSD-NH₂ | 20 (2) |
| (SEQ ID NO:29) | | |
| 36 | Ac-IESVSD-NH₂ | 5.3 (2) |
| (SEQ ID NO:30) | | |
| 37 | Ac-DSSSEI-NH₂ | 20 (2) |
| (SEQ ID NO:31) | | |
| 38 | Ac-iesssd-NH₂ | 20 (2) |
| (SEQ ID NO:32) | | |
| 39 | Ac-dsssei-NH₂ | 20 (2) |
| (SEQ ID NO:33) | | |
| 40 | Ac-IESGSD-NH₂ | |
| (SEQ ID NO:98) | | |
| 41 | Ac-IESYSD-NH₂ | |
| (SEQ ID NO:99) | | |
| 42 | H-RRMKWKKLTIESSSDEE-NH₂ | |
| (SEQ ID NO:100) | | |
| 43 | H-RQIKIWFQNRRMKWKKLTIESSSDEE-NH₂ | |
| (SEQ ID NO:101) | | |
| 44 | H-RRMKWKKIESSSD-NH₂ | |
| (SEQ ID NO:102) | | |
| 45 | Biotin-RRMKWKKLTIESSSDEE-NH₂ | |
| (SEQ ID NO:100) | | |
| 46 | Biotinyl-YGRKKRRQRRRGGGGLTIESSSDEE-NH₂ | |
| (SEQ ID NO:41) | | |
| 47 | Ac-TIESSS-NH₂ | 12 |
| (SEQ ID NO:34) | | |
| 48 | Ac-IESSSDEE-NH₂ | >50 |
| (SEQ ID NO:35) | | |
| 49 | Ac-IESSSD-NH₂ | 4.3 (5) |
| (SEQ ID NO:36) | | |
| 50 | Ac-ESSSDE-NH₂ | 11 |
| (SEQ ID NO:37) | | |
| 51 | Ac-SSSDEE-NH₂ | 22 |
| (SEQ ID NO:38) | | |
| 52 | Ac-SSSDE-NH₂ | 17 |
| (SEQ ID NO:39) | | |
| 53 | Ac-IESSSDE-NH₂ | 19 |
| (SEQ ID NO:40) | | |
| 54 | H-YGRKKRRQRRRGGGGLTIESSSDEE-NH₂ | 6.5 |
| (SEQ ID NO:41) | | |

Equivalents

Those skilled in the art will recognize, or be able to ascertain using no more than routine experimentation, many equivalents to the specific embodiments and methods described herein. Such equivalents are intended to be encompassed by the scope of the following claims.

What is claimed is:

1. A peptide compound of the formula
Y₁-(Xₐ)ₖ-(X₁¹)ₘ-(X₂¹)ₙ-(X₃¹)ₚ-(X₄¹)-(X₅¹)-(X₆¹)-(X₇¹)_{q}-(X₈¹)ᵣ-(X_{b})ₛ-Y₂ (I),
wherein
Y1 is hydrogen, alkyl or acyl;
Y2 is —OH or —NR₂, where each R is independently hydrogen or lower alkyl;
Xₐ and X_{b} are each, independently, a peptidic structure comprising from 1 to 25 amino acid residues;
X₁¹ is lysine, alanine, threonine, histidine, methionine or an analogue thereof;
X₂¹ is threonine, glutamic acid, alanine, isoleucine, valine or an analogue thereof;
X₃¹ is glutamic acid, alanine, proline, threonine or aspartic acid or an analogue thereof;
X₄¹ is serine, valine, glutamine or alanine or an analogue thereof;
X₅¹ is serine, alanine, phenylalanine, leucine, valine or an analogue thereof;
X₆¹ is serine or an analogue thereof;
X₇¹ is aspartic acid, glutamic acid, alanine, methionine, proline, valine or an analogue thereof;
X₈¹ is serine, threonine; phenylalanine, glutamic acid, isoleucine or an analogue thereof; and
k, m, n, p, q, r and s are each, independently, 0 or 1.

2. The peptide compound of claim 1, wherein k is 1, m is 1, n is 1, and p is 1.

3. The peptide compound of claim 1, wherein m is 1, n is 1, and p is 1.

4. The peptide compound of claim 1, wherein n is 1 and p is 1.

5. The peptide compound of claim 1, wherein when s is 1, q is 1, and r is 1.

6. The peptide compound of claim 1, wherein r is 1 and q is 1.

7. The peptide compound of claim 1, wherein one or more of X₁¹, X₂¹, X₃¹, X₄¹, X₅¹, X₆¹, X₇¹ and X₈¹ is conservatively substituted.

8. The peptide compound of claim 1, wherein X₆¹ is not conservatively substituted and at least one of X₄¹ and X₅¹ is serine.

9. The peptide compound of claim 1, wherein at least one of X₄¹ and X₅¹ is serine.

10. The peptide compound of claim 1, wherein
X¹ is threonine or histidine;
X₂¹ is threonine or isoleucine;
X₃¹ is glutamic acid or aspartic acid;
X₄¹ is serine;
X₅¹ is serine;
X₇¹ is aspartic acid; and
X₈¹ is glutamic acid.

11. The peptide compound of claim 1, wherein the peptide compound is between 10-20 amino acids in length.

12. The peptide compound of claim 1, wherein the peptide compound is between 15-30 amino acids in length.

13. The peptide compound of claim 1, wherein Y1 is hydrogen.

14. The peptide compound of claim 1, wherein Y1 is acyl.

15. The peptide compound of claim 1, wherein Y1 is alkyl.

16. A peptide compound comprising a structure selected from the group consisting of KVESSSVFSKPASVTVASDA (SEQ ID NO: 1), PAAIPPSLTDYSVPFHHTPVSSMSSDL (SEQ ID NO:2), LTIESSSDEEEDPPAKRKAIF (SEQ ID NO:3), KPASVTVASDASKKIDVIDL (SEQ ID NO:4), LTIESSSDEE (SEQ ID NO:5), FMSETQSSPTK (SEQ ID NO:6), SVPFHHTPVSSMSSDL (SEQ ID NO:7), SVPFHHTPVS (SEQ ID NO:8), TPVSSMSSDL (SEQ ID NO:9), LTIESSSDEE (SEQ ID NO:10), ATIESSSDEE (SEQ ID NO:11), LAIESSSDEE (SEQ ID NO:12), LTAESSSDEE (SEQ ID NO:13), LTIASSSDEE (SEQ ID NO:14), LTIEASSDEE (SEQ ID NO:15), LTIESASDEE (SEQ ID NO:16), LTIESSSAEE (SEQ ID NO:17), LTIESSSDAE (SEQ ID NO:18), LTIESSSDEA (SEQ ID NO:19), LTIESSSDE (SEQ ID NO:20), TIESSSDEE (SEQ ID NO:21), TIESSSDE (SEQ ID NO:22), TIESSSD (SEQ ID NO:23), LTIESSSDEE(SEQ ID NO:24), IES(B-Ala)SD (SEQ ID NO:25), IESASD (SEQ ID NO:26), IASASD (SEQ ID NO:27), IESFSD (SEQ ID NO:28), IESLSD (SEQ ID NO:29), IESVSD (SEQ ID NO:30), DSSSEI (SEQ ID NO:31), Iesssd (SEQ ID NO:32), Dsssei (SEQ ID NO:33), TIESSS (SEQ ID NO:34), IESSSDEE (SEQ ID NO:35), IESSSD (SEQ ID NO:36), ESSSDE (SEQ ID NO:37), SSSDEE(SEQ ID NO:38), SSSDE (SEQ ID NO:39), IESSSDE(SEQ ID NO:40), and YGRKKRRQRRRGGGGLTIESSSDEE(SEQ ID NO:41).

17. A peptide compound of the formula
Y₁-Xₐ-X₁²-X₂²-X₃²-X₄²-X₅²-X₆²-X₇²-X_{b}-Y₂ (II),
wherein
Y₁ is hydrogen, alkyl or acyl;
Y₂ is —OH, amino or monosubstituted or disubstituted amino;
Xₐ and X_{b} are each, independently, a direct bond or a peptidic structure comprising from 1 to about 25 amino acid residues;
X₁² is lysine, serine, phenylalanine, glycine or serine;
X₂² is serine, tyrosine, valine, alanine or glycine;
X₃² is any amino acid residue;
X₄² is serine, leucine, tyrosine, phenylalanine, asparagine or alanine;
X₅² is leucine, serine, phenylalanine or tyrosine;
X₆² is tryptophan; and
X₇² is proline.

18. The peptide compound of claim 17, wherein Y₁ is hydrogen.

19. The peptide compound of claim 17, wherein Y₁ is a C₁-C₆-alkyl.

20. The peptide compound of claim 17, wherein Y₁ is a C₁-C₆-acyl.

21. The peptide compound of claim 17, wherein Y₂ is —NR₂, where each R is independently hydrogen or alkyl.

22. The peptide compound of claim 21, wherein R is a C₁-C₆-alkyl.

23. The peptide compound of claim 17, wherein X₁² is serine.

24. The peptide compound of claim 17, wherein X₂² is serine, tyrosine or valine.

25. The peptide compound of claim 17, wherein X₃² is proline, isoleucine, serine, tryptophan, alanine, valine, tyrosine, or phenylalanine.

26. The peptide compound of claim 17, wherein X₄² is serine or leucine.

27. The peptide compound of claim 17, wherein X₅² is leucine.

28. The peptide compound of claim 17, wherein said compound comprises a structure selected from the group consisting of Y₁-Xₐ-Ser-Ser-X₃²-Ser-Leu-Trp-Pro-X_{b}-Y₂; Y₁-Xₐ-Ser-Tyr-X₃²-Ser-Leu-Trp-Pro-X_{b}-Y₂; Y₁-Xₐ-Ser-Val-X₃²-Ser-Leu-Trp-Pro-X_{b}-Y₂; Y₁-Xₐ-Ser-Ser-X₃²-Leu-Leu-Trp-Pro-X_{b}-Y₂; Y₁-Xₐ-Ser-Tyr-X₃²-Leu-Leu-Trp-Pro-X_{b}-Y₂; and Y₁-Xₐ-Ser-Val-X₃²-Leu-Leu-Trp-Pro-X_{b}-Y₂.

29. The peptide compound of claim 17, wherein said compound comprises a structure selected from the group consisting of Y₁-Xₐ-Gly-Ser-Pro-Ala-Leu-Trp-Pro-X_{b}-Y₂; Y₁-Xₐ-Leu-Val-Ile-Asp-Leu-Trp-Pro-X_{b}-Y₂; Y₁-Xₐ-Ser-Tyr-Ser-Phe-Leu-Trp-Pro-X_{b}-Y₂; Y₁-Xₐ-Ser-Tyr-Trp-Tyr-Leu-Trp-Pro-X_{b}-Y₂; Y₁-Xₐ-Phe-Tyr-Ala-Ser-Leu-Trp-Pro-X_{b}-Y₂; Y₁-Xₐ-Tyr-Ala-Ala-Ser-Phe-Trp-Pro-X_{b}-Y₂; Y₁-Xₐ-Phe-Ala-Val-Leu-Ser-Trp-Pro-X_{b}-Y₂; Y₁-Xₐ-Gly-Val-Tyr-Leu-Ser-Trp-Pro-X_{b}-Y₂; Y₁-Xₐ-Ser-Ser-Trp-Leu-Ser-Trp-Pro-X_{b}-Y₂; and Y₁-Xₐ-Ser-Gly-Phe-Ser-Tyr-Trp-Pro-X_{b}-Y₂.

30. A peptide compound comprising a structure selected from the group consisting of Ser-Met-Phe-Gly-Ser-Pro-Ala-Leu-Trp-Pro-Leu-Arg (SEQ ID NO:56); Leu-Val-Ile-Asp-Leu-Trp-Pro-Ala-Phe-Val-Arg (SEQ ID NO:57); Ser-Tyr-Ser-Phe-Leu-Trp-Pro-Ile-Val-Phe-Lys (SEQ ID NO:58); Glu-Leu-Phe-Ser-Tyr-Trp-Pro-Trp-Pro-Phe-Tyr-Arg (SEQ ID NO:59); Phe-Tyr-Ala-Ser-Leu-Trp-Pro-His-Leu-Tyr-Val (SEQ ID NO:60); Val-Gly-Thr-Gly-Ala-Ser-Ser-Tyr-Ala-Ala-Ser-Phe-Trp-Pro-Trp-Met-Thr-Tyr-Tyr-Trp (SEQ ID NO:61); Phe-Ala-Val-Leu-Ser-Trp-Pro-Leu-Tyr-Glu-Tyr (SEQ ID NO:62); Gly-Val-Tyr-Leu-Ser-Trp-Pro-Gly-Ser-Met-Ala (SEQ ID NO:63); Ser-Ser-Tyr-Ser-Ser-Trp-Leu-Ser-Trp-Pro-Arg (SEQ ID NO:64); and Ser-Gly-Phe-Ser-Tyr-Trp-Pro-Phe-Phe-Phe-Val (SEQ ID NO:65).

31. A peptide compound of the formula
Y₁-Xₐ-Ser-Pro-Asp-X₁³-X₂³-Ala-X_{b}-Y₂ (III),
wherein
Y₁ is hydrogen, alkyl or acyl;
Y₂ is —OH, amino or monosubstituted or disubstituted amino;
Xₐ and X_{b} are each, independently, a direct bond or a peptidic structure comprising from 1 to about 25 amino acid residues;
X₁³ is Tyr or Leu; and
X₂³ is Gln or Leu.

32. The peptide compound of claim 31, wherein Y₁ is hydrogen.

33. The peptide compound of claim 31, wherein Y₁ is a C₁-C₆-alkyl.

34. The peptide compound of claim 31, wherein Y₁ is a C₁-C₆-acyl.

35. The peptide compound of claim 31, wherein Y₂ is —NR₂, where each R is independently hydrogen or alkyl.

36. The peptide compound of claim 35, wherein R is a C₁-C₆-alkyl.

37. The peptide compound of claim 31, wherein said compound comprises a structure selected from the group consisting of Y₁-Xₐ-Ser-Pro-Asp-Tyr-Gln-Ala-X_{b}-Y₂ and Y₁-Xₐ-Ser-Pro-Asp-Leu-Leu-Ala-X_{b}-Y₂.

38. A peptide compound comprising a structure selected from the group consisting of Arg-Met-Val-Ser-Pro-Asp-Tyr-Gln-Ala-Thr-Ser-Pro (SEQ ID NO:66) and Leu-Ser-Phe-Ser-Pro-Asp-Leu-Leu-Ala-Leu-Arg-Gly-Met (SEQ ID NO:67).

39. A peptide compound of the formula
Y₁-Xₐ-Ser-Pro-Ala-Leu-Trp-X_{b}-Y₂ (IV),
wherein
Y₁ is hydrogen, alkyl or acyl;
Y₂ is —OH, amino or monosubstituted or disubstituted amino; and
Xₐ and X_{b} are each, independently, a direct bond or a peptidic structure comprising from 1 to about 25 amino acid residues.

40. A peptide compound comprising a structure selected from the group consisting of Ser-Met-Phe-Gly-Ser-Pro-Ala-Leu-Trp-Pro-Leu-Arg (SEQ ID NO:68) and Val-Ser-Pro-Ala-Leu-Trp-Ser-Ser-Leu-Arg-Gly (SEQ ID NO:69).

41. A peptide compound of the formula
Y₁-Xₐ-X₁⁵-X₂⁵-Trp-Leu-X₃⁵-Ser-X_{b}-Y₂ (V),
wherein
Y₁ is hydrogen, alkyl or acyl;
Y₂ is —OH, amino or monosubstituted or disubstituted amino;
Xₐ and X_{b} are each, independently, a direct bond or a peptidic structure comprising from 1 to about 25 amino acid residues;
X₁⁵ is Ser or Pro;
X₂⁵ is Ser or Trp; and
X₃⁵ is Phe or Ala.

42. The peptide compound of claim 41, wherein X₁⁵ is Ser.

43. The peptide compound of claim 41, wherein X₂⁵ is Ser.

44. The peptide compound of claim 41, wherein X₃⁵ is Ala.

45. The peptide compound of claim 41, wherein said compound comprises a structure selected from the group consisting of Y₁-Xₐ-Ser-Ser-Trp-Leu-Ala-Ser-X_{b}-Y₂; Y₁-Xₐ-Ser-Trp-Trp-Leu-Ala-Ser-X_{b}-Y₂ and Y₁-Xₐ-Pro-Ser-Trp-Leu-Ala-Ser-X_{b}-Y₂.

46. A peptide compound comprising a structure selected from the group consisting of Gly Phe Val Ser Ser Trp Leu Phe Ser Ala Ser (SEQ ID NO:70); Ala Ser Met Ser Trp Trp Leu Ala Ser Ser Pro (SEQ ID NO:71); and Phe Val His Pro Ser Trp Leu Ala Ser Phe Leu (SEQ ID NO:72).

47. A peptide compound of the formula
Y₁-Xₐ-Phe-X₁⁶-X₂⁶-X₃⁶-X₄⁶-X₅⁶-X₆⁶-X_{b}-Y₂ (VI),
wherein Y₁ is hydrogen, alkyl or acyl;
Y₂ is —OH, amino or monosubstituted or disubstituted amino;
Xₐ and X_{b} are each, independently, a direct bond or a peptidic structure comprising from 1 to about 25 amino acid residues;
X₁⁶ is Ser or Val;
X₂⁶ is His, Thr, Gly or Phe;
X₃⁶ is Pro, Ile, or Tyr;
X₄⁶ is Ser, Tyr, Ala, Gly or Cys;
X₅⁶ is Trp or Gly; and
X₆⁶ is Leu, Ser, Tyr or Arg.

48. The peptide compound of claim 47, wherein X₁⁶ is Ser.

49. The peptide compound of claim 47, wherein X₂⁶ is His or Thr.

50. The peptide compound of claim 47, wherein X₃⁶ is Pro.

51. The peptide compound of claim 47, wherein X₄⁶ is Ala.

52. The peptide compound of claim 47, wherein X₅⁶ is Trp.

53. The peptide compound of claim 47, wherein X₆⁶ is Ser.

54. The peptide compound of claim 47, wherein said compound comprises a structure selected from the group consisting of Y₁-Xₐ-Phe-Ser-His-Pro-Ala-Trp-Ser-X_{b}-Y₂; Y₁ -Xₐ-Phe-Ser-Thr-Pro-Ala-Trp-Ser-X_{b}-Y₂; Y₁-Xₐ-Phe-Val-His-Pro-Ser-Trp-Leu-X_{b}-Y₂; Y₁-Xₐ-Phe-Ser-His-Pro-Tyr-Trp-Ser-X_{b}-Y₂; Y₁-Xₐ-Phe-Ser-Gly-Pro-Ala-Trp-Ser-X_{b}-Y₂; Y₁-Xₐ-Phe-Ser-Phe-Ile-Ala-Trp-Ser-X_{b}-Y₂; Y₁-Xₐ-Phe-Ser-Thr-Tyr-Gly-Trp-Tyr-X_{b}-Y₂; and Y₁-Xₐ-Phe-Ser-Thr-Pro-Cys-Gly-Arg-X_{b}-Y₂.

55. A peptide compound comprising a structure selected from the group consisting of Phe Val His Pro Ser Trp Leu Ala Ser Phe Leu (SEQ ID NO:73); Phe Ser His Pro Tyr Trp Ser Tyr Leu Phe Ser (SEQ ID NO:74); Phe Ser Gly Pro Ala Trp Ser Leu His Lys His (SEQ ID NO:75); Phe Ser Phe Ile Ala Trp Ser Pro Ala Met Leu (SEQ ID NO:76); Phe Ser Thr Tyr Gly Trp Tyr Ser Pro Phe His (SEQ ID NO:77); or Ile Pro Phe Ser Thr Pro Cys Gly Arg Trp Cys (SEQ ID NO:78).

56. A peptide compound of the formula
Y₁-Xₐ-X₁⁷-X₂⁷-X₃⁷-X₄⁷-X₅⁷-X₆⁷-X₇⁷-X₈⁷-X₉⁷-X_{b}-Y₂ (VII)
wherein Y₁ is hydrogen, alkyl or acyl;
Y₂ is —OH, amino or monosubstituted or disubstituted amino;
Xₐ and X_{b} are each, independently, a direct bond or a peptidic structure comprising from 1 to about 25 amino acid residues;
X₁⁷ is Ser, Trp, Phe, Ala or Val;
X₂⁷ is Ser, Leu, Pro, Trp or Val;
X₃⁷ is Ser, Tyr, Val, Met or His;
X₄⁷ is Pro, Ser, Leu, Arg, Tyr, Val or Trp;
X₅⁷ is Phe, Ser, Trp, Arg or Leu;
X₆⁷ is Trp, Asn, Met, Asp, Gly, Phe;
X₇⁷ is Ala, Trp, Leu or Ser;
X₈⁷ is Arg, Pro, Phe, Ala, Ser, Tyr; and
X₉⁷ is Pro, Asn, Ser, Trp, Thr, Phe, Ala.

57. The peptide compound of claim 56, wherein X₁⁷ is Ser.

58. The peptide compound of claim 56, wherein X₂⁷ is Ser.

59. The peptide compound of claim 56, wherein X₃⁷ is Ser or Tyr.

60. The peptide compound of claim 56, wherein X₄⁷ is Pro.

61. The peptide compound of claim 56, wherein X₆⁷ is Trp.

62. The peptide compound of claim 56, wherein X₇⁷ is Leu.

63. The peptide compound of claim 56, wherein X₈⁷ is Ala.

64. The peptide compound of claim 56, wherein X₉⁷ is Ser.

65. The peptide compound of claim 56, wherein said compound comprises a structure selected from the group consisting of Y₁-Xₐ-Ser-Ser-Ser-Pro-Trp-Trp-Leu-Ala-Ser-Xb-Y2; Y1-Xa-Ser-Ser-Ser-Pro-Phe-Trp-Leu-Ala-Ser-Xb-Y2; Y1-Xa-Ser-Ser-Ser-Pro-Ser-Trp-Leu-Ala-Ser-Xb-Y2; Y1-Xa-Ser-Ser-Tyr-Pro-Trp-Trp-Leu-Ala-Ser-Xb-Y2; Y1-Xa-Ser-Ser-Tyr-Pro-Phe-Trp-Leu-Ala-Ser-Xb-Y2; Y1-Xₐ-Ser-Ser-Tyr-Pro-Ser-Trp-Leu-Ala-Ser-Xb-Y2; Y1-Xa-Ser-Ser-Val-Pro-Phe-Trp-Ala-Arg-Pro-Xb-Y2; Y1-Xₐ-Ser-Leu-Val-Pro-Phe-Asn-Trp-Pro-Asn-X_{b}-Y₂; Y₁-Xₐ-Ser-Pro-Tyr-Pro-Ser-Met-Leu-Phe-Ser-X_{b}-Y₂; Y₁-Xₐ-Trp-Trp-Tyr-Pro-Trp-Asp-Leu-Ala-Ser-X_{b}-Y₂; Yl-Xₐ-Phe-Val-His-Pro-Ser-Trp-Leu-Ala-Ser-X_{b}-Y₂; Y₁-Xₐ-Ala-Ser-Met-Ser-Trp-Trp-Leu-Ala-Ser-X_{b}-Y₂; Y₁-Xₐ-Ser-Ser-Tyr-Ser-Ser-Trp-Leu-Ser-Trp-X_{b}-Y₂; Y₁-Xₐ-Ser-Ser-Ser-Leu-Phe-Trp-Ser-Ala-Thr-X_{b}-Y₂; Y₁-Xₐ-Ser-Ser-Ser-Arg-Ser-Trp-Ala-Ala-Phe-X_{b}-Y₂; Y₁-Xₐ-Ser-Ser-Ser-Tyr-Trp-Gly-Leu-Tyr-Pro-X_{b}-Y₂; Y₁-Xₐ-Ser-Ser-Tyr-Val-Arg-Trp-Leu-Ala-Ala-X_{b}-Y₂; and Y₁-Xₐ-Val-Ser-Ser-Trp-Leu-Phe-Ser-Ala-Ser-X_{b}-Y₂.

66. A peptide compound comprising a structure selected from the group consisting of Ser-Ser-Val-Pro-Phe-Trp-Ala-Arg-Pro-Leu-Val (SEQ ID NO:79); Ser-Leu-Ser-Ser-Leu-Val-Pro-Phe-Asn-Trp-Pro-Asn-Leu-Phe-Ser-Trp-Arg-Tyr-Ser-Trp (SEQ ID NO:80); Leu-Gly-Ser-Pro-Tyr-Pro-Ser-Met-Leu-Phe-Ser-Asp-His (SEQ ID NO:81); Phe-Trp-Trp-Tyr-Pro-Trp-Asp-Leu-Ala-Ser-Tyr (SEQ ID NO:82); Phe-Val-His-Pro-Ser-Trp-Leu-Ala-Ser-Phe-Leu (SEQ ID NO:83); Ala-Ser-Met-Ser-Trp-Trp-Leu-Ala-Ser-Ser-Pro (SEQ ID NO:84); Ser-Ser-Tyr-Ser-Ser-Trp-Leu-Ser-Trp-Pro-Arg (SEQ ID NO:85); Ser-Ser-Ser-Leu-Phe-Trp-Ser-Ala-Thr-Ser-Arg (SEQ ID NO:86); Ser-Ser-Ser-Arg-Ser-Trp-Ala-Ala-Phe-Glu-His (SEQ ID NO:87); Ser-Ser-Ser-Tyr-Trp-Gly-Leu-Tyr-Pro-Ser-Leu-Ser-Leu (SEQ ID NO:88); Ser-Ser-Tyr-Val-Arg-Trp-Leu-Ala-Ala-Ala-Gln (SEQ ID NO:89); Gly-Phe-Val-Ser-Ser-Trp-Leu-Phe-Ser-Ala-Ser (SEQ ID NO:90); and Ser-His-Gly-Trp-Phe-Trp-Ser-Ser-Ser-Gln-Gly (SEQ ID NO:91).

67. A peptide compound of the formula
Y₁-Xₐ-Cys-X₁⁸-X₂⁸-X₃⁸-X₄⁸-Gly-X₅⁸-X₆⁸-X₇⁸-Cys-X_{b}-Y₂ (VIII),
wherein Y₁ is hydrogen, alkyl or acyl;
Y₂ is —OH, amino or monosubstituted or disubstituted amino;
Xₐ and X_{b} are each, independently, a direct bond or a peptidic structure comprising from 1 to about 25 amino acid residues;
X₁⁸ is an aromatic amino acid residue or threonine;
X₂⁸ is Gly, Phe, Gln, Arg, Met, Trp;
X₃⁸ is Asp or Glu;
X₄⁸ is Glu or Asp;
X₅⁸ is Tyr or Trp;
X₆⁸ is Pro, Trp, Thr, Leu, Phe, Tyr, Met; and
X₇⁸ is His, Asp, Ser, Ala, Leu, Met, Trp.

68. The peptide compound of claim 67, wherein said compound comprises a structure selected from the group consisting of Y₁-Xₐ-Cys-Trp-Gly-Asp-Asp-Gly-Trp-Pro-Ala-His-Cys-X_{b}-Y₂; Y₁-Xₐ-Cys-Phe-Phe-Asp-Asp-Gly-Tyr-Trp-Trp-Asp-Cys-X_{b}-Y₂; Y₁-Xₐ-Cys-Tyr-Gln-Asp-Asp-Gly-Tyr-Thr-Val-Ser-Cys-X_{b}-Y₂; Y₁-Xₐ-Cys-Tyr-Met-Glu-Asp-Gly-Tyr-Leu-Trp-Ala-Cys-X_{b}-Y₂; Y₁-Xₐ-Cys-Phe-Phe-Glu-Asp-Gly-Tyr-Phe-His-Ala-Cys-X_{b}-Y₂; Y₁-Xₐ-Cys-Tyr-Gly-Asp-Asp-Gly-Trp-Phe-Met-Leu-Cys-X_{b}-Y₂; Y₁-Xₐ-Cys-Tyr-Met-Asp-Glu-Gly-Trp-Tyr-Tyr-Ser-Cys-X_{b}-Y₂; Y₁-Xₐ-Cys-Trp-Gln-Glu-Asp-Gly-Trp-Leu-Tyr-Leu-Cys-X_{b}-Y₂; Y₁-Xₐ-Cys-Trp-Arg-Glu-Asp-Gly-Tyr-Trp-Trp-Trp-Cys-X_{b}-Y₂; Y₁-Xₐ-Cys-Trp-Gln-Asp-Asp-Gly-Trp-Tyr-Tyr-Met-Cys-X_{b}-Y₂; Y₁-Xₐ-Cys-Tyr-Trp-Asp-Asp-Gly-Trp-Met-Leu-Glu-Cys-X_{b}-Y₂; Y₁-Xₐ-Cys-Trp-Arg-Glu-Asp-Gly-Tyr-Trp-Trp-Trp-Cys-X_{b}-Y₂; and Y₁-Xₐ-Cys-Thr-Trp-Asp-Asp-Gly-Trp-Met-Phe-Leu-Cys-X_{b}-Y₂.

69. A nucleic acid molecule encoding the peptide compound of any one of claims 1, 17, 31, 39, 41, 47, 56, or 67.

70. A cell comprising the nucleic acid molecule of any one of claims 1, 17, 31, 39, 41, 47, 56, or 67.

71. A pharmaceutical composition comprising a therapeutically effective amount of the peptide compound of any one of claims 1, 17, 31, 39, 41, 47, 56, or 67 and a pharmaceutically acceptable carrier.

72. A method of treating a subject suffering from an androgen-associated disorder, comprising administering to the subject a therapeutically effective amount of the peptide compound of any one of claims 1, 17, 31, 39, 41, 47, 56, or 67, thereby treating a subject suffering from an androgen-associated disorder.

73. The method of claim 72, wherein the peptide compound is cell permeable.

74. The method of claim 73, wherein the peptide compound further comprises an amino acid sequence which facilitates passage of the peptide across the cell membrane.

75. The method of claim 74, wherein the amino acid sequence which facilitates passage of the peptide across the cell membrane comprises the Kaposi FGF signal sequence.

76. The method of claim 74, wherein the amino acid sequence which facilitates passage of the peptide across the cell membrane comprises sequences derived from the HIV TAT protein.

77. The method of claim 74, wherein the amino acid sequence which facilitates passage of the peptide across the cell membrane comprises the antennapedia homeodomain.

78. The method of claim 72, wherein the androgen-associated disorder is prostate cancer.

79. The method of claim 72, wherein the androgen-associated disorder is colon cancer.

80. The method of claim 72, wherein the androgen-associated disorder is lung cancer.

81. The method of claim 72, wherein the androgen-associated disorder is benign prostatic hypertrophy.

82. The method of claim 72, wherein the androgen-associated disorder is acne.

83. The method of claim 72, wherein the androgen-associated disorder is hirsutism.

84. A method of identifying a compound that binds to an androgen receptor or a fragment thereof, comprising:
contacting the androgen receptor or a fragment thereof with a peptide compound of any one of claims 1, 17, 31, 39, 41, 47, 56, or 67, thereby forming a peptide compound-androgen receptor complex;
contacting the peptide compound-androgen receptor complex with a test compound; and
determining if the peptide compound dissociates from the peptide compound-androgen receptor complex in the presence of the test compound, thereby identifying a compound that binds to the androgen receptor.

85. The method of claim 84, wherein the androgen receptor or a fragment thereof comprises the androgen receptor DNA binding domain.

86. The method of claim 84, wherein the androgen receptor or a fragment thereof is linked to a solid support.

87. The method of claim 84, wherein the solid support is a chromatography column.


SEQUENCE LISTING (102 sequences)

SEQ ID NO: 1 - protein, 20 aa, Artificial Sequence
  Androgen Receptor Binding Polypeptides
KVESSSVFSKPASVTVASDA

SEQ ID NO: 2 - protein, 27 aa, Artificial Sequence
  Androgen Receptor Binding Polypeptides
PAAIPPSLTDYSVPFHHTPVSSMSSDL

SEQ ID NO: 3 - protein, 21 aa, Artificial Sequence
  Androgen Receptor Binding Polypeptides
LTIESSSDEEEDPPAKRKAIF

SEQ ID NO: 4 - protein, 20 aa, Artificial Sequence
  Androgen Receptor Binding Polypeptides
KPASVTVASDASKKIDVIDL

SEQ ID NO: 5 - protein, 10 aa, Artificial Sequence
  Androgen Receptor Binding Polypeptides
LTIESSSDEE

SEQ ID NO: 6 - protein, 11 aa, Artificial Sequence
  Androgen Receptor Binding Polypeptides
FMSETQSSPTK

SEQ ID NO: 7 - protein, 16 aa, Artificial Sequence
  Androgen Receptor Binding Polypeptides
SVPFHHTPVSSMSSDL

SEQ ID NO: 8 - protein, 10 aa, Artificial Sequence
  Androgen Receptor Binding Polypeptides
SVPFHHTPVS

SEQ ID NO: 9 - protein, 10 aa, Artificial Sequence
  Androgen Receptor Binding Polypeptides
TPVSSMSSDL

SEQ ID NO: 10 - protein, 10 aa, Artificial Sequence
  Androgen Receptor Binding Polypeptides
LTIESSSDEE

SEQ ID NO: 11 - protein, 10 aa, Artificial Sequence
  Androgen Receptor Binding Polypeptides
ATIESSSDEE

SEQ ID NO: 12 - protein, 10 aa, Artificial Sequence
  Androgen Receptor Binding Polypeptides
LAIESSSDEE

SEQ ID NO: 13 - protein, 10 aa, Artificial Sequence
  Androgen Receptor Binding Polypeptides
LTAESSSDEE

SEQ ID NO: 14 - protein, 10 aa, Artificial Sequence
  Androgen Receptor Binding Polypeptides
LTIASSSDEE

SEQ ID NO: 15 - protein, 10 aa, Artificial Sequence
  Androgen Receptor Binding Polypeptides
LTIEASSDEE

SEQ ID NO: 16 - protein, 10 aa, Artificial Sequence
  Androgen Receptor Binding Polypeptides
LTIESASDEE

SEQ ID NO: 17 - protein, 10 aa, Artificial Sequence
  Androgen Receptor Binding Polypeptides
LTIESSSAEE

SEQ ID NO: 18 - protein, 10 aa, Artificial Sequence
  Androgen Receptor Binding Polypeptides
LTIESSSDAE

SEQ ID NO: 19 - protein, 10 aa, Artificial Sequence
  Androgen Receptor Binding Polypeptides
LTIESSSDEA

SEQ ID NO: 20 - protein, 9 aa, Artificial Sequence
  Androgen Receptor Binding Polypeptides
LTIESSSDE

SEQ ID NO: 21 - protein, 9 aa, Artificial Sequence
  Androgen Receptor Binding Polypeptides
TIESSSDEE

SEQ ID NO: 22 - protein, 8 aa, Artificial Sequence
  Androgen Receptor Binding Polypeptides
TIESSSDE

SEQ ID NO: 23 - protein, 7 aa, Artificial Sequence
  Androgen Receptor Binding Polypeptides
TIESSSD

SEQ ID NO: 24 - protein, 10 aa, Artificial Sequence
  Androgen Receptor Binding Polypeptides
LTIESSSDEE

SEQ ID NO: 25 - protein, 6 aa, Artificial Sequence
  Androgen Receptor Binding Polypeptides
IESXSD

SEQ ID NO: 26 - protein, 6 aa, Artificial Sequence
  Androgen Receptor Binding Polypeptides
IESASD

SEQ ID NO: 27 - protein, 6 aa, Artificial Sequence
  Androgen Receptor Binding Polypeptides
IASASD

SEQ ID NO: 28 - protein, 6 aa, Artificial Sequence
  Androgen Receptor Binding Polypeptides
IESFSD

SEQ ID NO: 29 - protein, 6 aa, Artificial Sequence
  Androgen Receptor Binding Polypeptides
IESLSD

SEQ ID NO: 30 - protein, 6 aa, Artificial Sequence
  Androgen Receptor Binding Polypeptides
IESVSD

SEQ ID NO: 31 - protein, 6 aa, Artificial Sequence
  Androgen Receptor Binding Polypeptides
DSSSEI

SEQ ID NO: 32 - protein, 6 aa, Artificial Sequence
  Androgen Receptor Binding Polypeptides
IESSSD

SEQ ID NO: 33 - protein, 6 aa, Artificial Sequence
  Androgen Receptor Binding Polypeptides
DSSSEI

SEQ ID NO: 34 - protein, 6 aa, Artificial Sequence
  Androgen Receptor Binding Polypeptides
TIESSS

SEQ ID NO: 35 - protein, 8 aa, Artificial Sequence
  Androgen Receptor Binding Polypeptides
IESSSDEE

SEQ ID NO: 36 - protein, 6 aa, Artificial Sequence
  Androgen Receptor Binding Polypeptides
IESSSD

SEQ ID NO: 37 - protein, 6 aa, Artificial Sequence
  Androgen Receptor Binding Polypeptides
ESSSDE

SEQ ID NO: 38 - protein, 6 aa, Artificial Sequence
  Androgen Receptor Binding Polypeptides
SSSDEE

SEQ ID NO: 39 - protein, 5 aa, Artificial Sequence
  Androgen Receptor Binding Polypeptides
SSSDE

SEQ ID NO: 40 - protein, 7 aa, Artificial Sequence
  Androgen Receptor Binding Polypeptides
IESSSDE

SEQ ID NO: 41 - protein, 25 aa, Artificial Sequence
  Androgen Receptor Binding Polypeptides
YGRKKRRQRRRGGGGLTIESSSDEE

SEQ ID NO: 42 - DNA, 15 bp, Artificial Sequence
  Androgen Responsive Element
agtacgtgatgttct

SEQ ID NO: 43 - DNA, 15 bp, Artificial Sequence
  Androgen Responsive Element
gaaacagcctgttct

SEQ ID NO: 44 - DNA, 15 bp, Artificial Sequence
  Androgen Responsive Element
agcacttgctgttct

SEQ ID NO: 45 - DNA, 15 bp, Artificial Sequence
  Androgen Responsive Element
atagcatcttgttct

SEQ ID NO: 46 - DNA, 15 bp, Artificial Sequence
  Androgen Responsive Element
agtcccacttgttct

SEQ ID NO: 47 - DNA, 15 bp, Artificial Sequence
  Androgen Responsive Element
agtacttgttgttct

SEQ ID NO: 48 - DNA, 15 bp, Artificial Sequence
  Androgen Responsive Element
agctcagcttgtact

SEQ ID NO: 49 - DNA, 15 bp, Artificial Sequence
  Androgen Responsive Element
agaacaacctgttga

SEQ ID NO: 50 - DNA, 15 bp, Artificial Sequence
  Androgen Responsive Element
tgaagttcctgttct

SEQ ID NO: 51 - DNA, 17 bp, Artificial Sequence
  Androgen Responsive Element
gtaaagtactccaagaa

SEQ ID NO: 52 - DNA, 15 bp, Artificial Sequence
  Androgen Responsive Element
ggaacagcaagtgct

SEQ ID NO: 53 - DNA, 17 bp, Artificial Sequence
  Androgen Responsive Element
ggaatacannntgttct

SEQ ID NO: 54 - DNA, 25 bp, Artificial Sequence
  Oligonucleotide
agtctggtacagggtgttcttttta

SEQ ID NO: 55 - DNA, 25 bp, Artificial Sequence
  Oligonucleotide
taaaaagaacaccctgtaccagact

SEQ ID NO: 56 - protein, 12 aa, Artificial Sequence
  Androgen Receptor Binding Polypeptides
SMFGSPALWPLR

SEQ ID NO: 57 - protein, 11 aa, Artificial Sequence
  Androgen Receptor Binding Polypeptides
LVIDLWPAFVR

SEQ ID NO: 58 - protein, 11 aa, Artificial Sequence
  Androgen Receptor Binding Polypeptides
SYSFLWPIVFK

SEQ ID NO: 59 - protein, 12 aa, Artificial Sequence
  Androgen Receptor Binding Polypeptides
ELFSYWPWPFYR

SEQ ID NO: 60 - protein, 11 aa, Artificial Sequence
  Androgen Receptor Binding Polypeptides
FYASLWPHLYV

SEQ ID NO: 61 - protein, 20 aa, Artificial Sequence
  Androgen Receptor Binding Polypeptides
VGTGASSYAASFWPWMTYYW

SEQ ID NO: 62 - protein, 11 aa, Artificial Sequence
  Androgen Receptor Binding Polypeptides
FAVLSWPLYEY

SEQ ID NO: 63 - protein, 11 aa, Artificial Sequence
  Androgen Receptor Binding Polypeptides
GVYLSWPGSMA

SEQ ID NO: 64 - protein, 11 aa, Artificial Sequence
  Androgen Receptor Binding Polypeptides
SSYSSWLSWPR

SEQ ID NO: 65 - protein, 11 aa, Artificial Sequence
  Androgen Receptor Binding Polypeptides
SGFSYWPFFFV

SEQ ID NO: 66 - protein, 12 aa, Artificial Sequence
  Androgen Receptor Binding Polypeptides
RMVSPDYQATSP

SEQ ID NO: 67 - protein, 13 aa, Artificial Sequence
  Androgen Receptor Binding Polypeptides
LSFSPDLLALRGM

SEQ ID NO: 68 - protein, 12 aa, Artificial Sequence
  Androgen Receptor Binding Polypeptides
SMFGSPALWPLR

SEQ ID NO: 69 - protein, 11 aa, Artificial Sequence
  Androgen Receptor Binding Polypeptides
VSPALWSSLRG

SEQ ID NO: 70 - protein, 11 aa, Artificial Sequence
  Androgen Receptor Binding Polypeptides
GFVSSWLFSAS

SEQ ID NO: 71 - protein, 11 aa, Artificial Sequence
  Androgen Receptor Binding Polypeptides
ASMSWWLASSP

SEQ ID NO: 72 - protein, 11 aa, Artificial Sequence
  Androgen Receptor Binding Polypeptides
FVHPSWLASFL

SEQ ID NO: 73 - protein, 11 aa, Artificial Sequence
  Androgen Receptor Binding Polypeptides
FVHPSWLASFL

SEQ ID NO: 74 - protein, 11 aa, Artificial Sequence
  Androgen Receptor Binding Polypeptides
FSHPYWSYLFS

SEQ ID NO: 75 - protein, 11 aa, Artificial Sequence
  Androgen Receptor Binding Polypeptides
FSGPAWSLHKH

SEQ ID NO: 76 - protein, 11 aa, Artificial Sequence
  Androgen Receptor Binding Polypeptides
FSFIAWSPAML

SEQ ID NO: 77 - protein, 11 aa, Artificial Sequence
  Androgen Receptor Binding Polypeptides
FSTYGWYSPFH

SEQ ID NO: 78 - protein, 11 aa, Artificial Sequence
  Androgen Receptor Binding Polypeptides
IPFSTPCGRWC

SEQ ID NO: 79 - protein, 11 aa, Artificial Sequence
  Androgen Receptor Binding Polypeptides
SSVPFWARPLV

SEQ ID NO: 80 - protein, 20 aa, Artificial Sequence
  Androgen Receptor Binding Polypeptides
SLSSLVPFNWPNLFSWRYSW

SEQ ID NO: 81 - protein, 13 aa, Artificial Sequence
  Androgen Receptor Binding Polypeptides
LGSPYPSMLFSDH

SEQ ID NO: 82 - protein, 11 aa, Artificial Sequence
  Androgen Receptor Binding Polypeptides
FWWYPWDLASY

SEQ ID NO: 83 - protein, 11 aa, Artificial Sequence
  Androgen Receptor Binding Polypeptides
FVHPSWLASFL

SEQ ID NO: 84 - protein, 11 aa, Artificial Sequence
  Androgen Receptor Binding Polypeptides
ASMSWWLASSP

SEQ ID NO: 85 - protein, 11 aa, Artificial Sequence
  Androgen Receptor Binding Polypeptides
SSYSSWLSWPR

SEQ ID NO: 86 - protein, 11 aa, Artificial Sequence
  Androgen Receptor Binding Polypeptides
SSSLFWSATSR

SEQ ID NO: 87 - protein, 11 aa, Artificial Sequence
  Androgen Receptor Binding Polypeptides
SSSRSWAAFEH

SEQ ID NO: 88 - protein, 13 aa, Artificial Sequence
  Androgen Receptor Binding Polypeptides
SSSYWGLYPSLSL

SEQ ID NO: 89 - protein, 11 aa, Artificial Sequence
  Androgen Receptor Binding Polypeptides
SSYVRWLAAAQ

SEQ ID NO: 90 - protein, 11 aa, Artificial Sequence
  Androgen Receptor Binding Polypeptides
GFVSSWLFSAS

SEQ ID NO: 91 - protein, 11 aa, Artificial Sequence
  Androgen Receptor Binding Polypeptides
SHGWFWSSSQG

SEQ ID NO: 92 - protein, 21 aa, Artificial Sequence
  Androgen Receptor Binding Polypeptides
EDPPAKRKAIFMSETQSSPTK

SEQ ID NO: 93 - protein, 21 aa, Artificial Sequence
  Androgen Receptor Binding Polypeptides
FMSETQSSPTKGVLMYQPSSV

SEQ ID NO: 94 - protein, 20 aa, Artificial Sequence
  Androgen Receptor Binding Polypeptides
KGVLMYQSSVRVPSVTSVDP

SEQ ID NO: 95 - protein, 21 aa, Artificial Sequence
  Androgen Receptor Binding Polypeptides
VRVPSVTSVDPAAIPPSLTDY

SEQ ID NO: 96 - protein, 9 aa, Artificial Sequence
  Androgen Receptor Binding Polypeptides
KVESSSVFS

SEQ ID NO: 97 - protein, 6 aa, Artificial Sequence
  Androgen Receptor Binding Polypeptides
LTIESS

SEQ ID NO: 98 - protein, 6 aa, Artificial Sequence
  Androgen Receptor Binding Polypeptides
IESGSD

SEQ ID NO: 99 - protein, 6 aa, Artificial Sequence
  Androgen Receptor Binding Polypeptides
IESYSD

SEQ ID NO: 100 - protein, 17 aa, Artificial Sequence
  Androgen Receptor Binding Polypeptides
RRMKWKKLTIESSSDEE

SEQ ID NO: 101 - protein, 26 aa, Artificial Sequence
  Androgen Receptor Binding Polypeptides
RQIKIWFQNRRMKWKKLTIESSSDEE

SEQ ID NO: 102 - protein, 13 aa, Artificial Sequence
  Androgen Receptor Binding Polypeptides
RRMKWKKIESSSD